(19) United States
(12) Patent Application Publication
(10) Pub. No.: US 2022/0089547 A1
(43) Pub. Date: Mar. 24, 2022

(54) COMPOUNDS FOR USE AS THERAPEUTICALLY ACTIVE SUBSTANCES IN THE TREATMENT AND/OR PREVENTION OF NEURORETINAL DISEASES

(71) Applicant: ENDOGENA THERAPEUTICS, INC., San Francisco, CA (US)

(72) Inventors: Matthias STEGER, Zurich (CH); Alex MUELLER, Zurich (CH); Mauro MARIGO, Zurich (CH); Bernhard FASCHING, Saint-Louis (FR); Daphna MOKADY, Toronto (CA)

(73) Assignee: ENDOGENA THERAPEUTICS, INC., San Francisco, CA (US)

(21) Appl. No.: 17/418,057

(22) PCT Filed: Dec. 27, 2019
(86) PCT No.: PCT/US2019/068759

§ 371 Date: Jun. 24, 2021

Related U.S. Application Data
(63) Continuation of application No. 16/235,543, filed on Dec. 28, 2018, now Pat. No. 10,752,593.
Application No. 16/235,543 is a continuation of application No. 10/752,593, now abandoned.

(51) Int. Cl. C07D 233/64; A61P 27/02; A61K 9/00; C07D 213/56

(52) U.S. Cl. CPC C07D 233/64; A61P 27/02; C07D 213/56; A61K 9/0048; A61K 9/0019

(57) ABSTRACT

A compound of the formula (I)

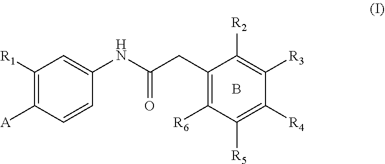

or a pharmaceutically acceptable salt thereof, wherein: A is a 5-oxazolyl residue or a pyridine-4-yl residue, R₁ is selected from fluoro and chloro; R₂, R₃, R₄, R₅ and R₆ of the phenyl ring B are independently from each other selected from hydrogen, a linear or branched alkyl having 1 to 4 carbon atoms, trifluoromethyl, 2,2,2-trifluoroethyl, methylsulfanyl, ethylsulfanyl, methylsulfonyl, ethylsulfonyl, difluoromethoxy, trifluoromethoxy, fluoro, bromo, chloro, methoxy, ethoxy, propoxy, butoxy, hydroxy and amino; and at least two of R₂, R₃, R₄, R₅ and R₆ are hydrogens, with the proviso that if R₁ is chloro, R₅ is not methoxy. Said compounds are useful as therapeutically active substances in the treatment and/or prevention of neuroretinal diseases, and in particular in the treatment and/or prevention of neuroretinal diseases leading to photoreceptor loss or degeneration of the outer retina.

The present invention relates to compounds for use as therapeutically active substances in the treatment and/or prevention of neuroretinal diseases, and in particular in the treatment and/or prevention of neuroretinal diseases leading to photoreceptor loss or degeneration of the outer retina.

The main feature of neurodegenerative diseases is an increasing loss of nerve cells, resulting in various neurological symptoms. The diseases can arise in different periods of life, which proceed diffusely or generalized and produce specific patterns of damage.

Of particular importance are neurodegenerative diseases of the eye. The retinal degeneration is a decay of the retina, which can finally result in the death of the cells of the retina. One of the most important forms of the retina degeneration is the so-called retinitis pigmentosa (RP) or also referred to as retinopathia pigmentosa. The chief function of the retina is transduction of light into nervous impulses by the rods and the cones. Retinitis pigmentosa is a chronic retinal degeneration where the deterioration is accompanied by abnormal deposits of pigment in the rods of the retina. The disease causes a progressive decrease in peripheral vision leading to malfunction of the side vision. Eventually, the person with retinitis pigmentosa can see only straight ahead so that the patient experiences a condition known as “tunnel vision”.

The therapeutic strategies for treating loss of vision caused by retinal cell damage vary, but they are all directed to controlling the illness causing the damage rather than reversing the damage caused by an illness by restoring or regenerating retinal cells.

WO 2016/073931 discloses a method for the treatment of retinitis pigmentosa in a human that comprises administering to the human a therapeutically effective amount of N-acetylcysteine amide (NACA) which reduces cone cell death in the eye.

EP 2 734 202 discloses a pharmaceutical composition containing 4-bromo-N-(imidazolidin-2-ylidene)-1H-benzimidazol-5-amine as active ingredient for modulating the alpha 2 adrenergic receptors. It was shown that said compound reduced and protected the retina from the damage caused by blue light.

US 2015/290215 discloses a composition comprising clozapine, n-desmethyl clozapine, olanzapine or derivatives thereof for treating a retinal disorder, which is caused by oxidative stress.

US 2016/0213671 relates to a pharmaceutical composition for the treatment or prophylaxis of a neurodegenerative disease, which is not based on a protein-folding disorder comprising as the active agent an inhibitor of the valosin-containing protein (VCP inhibitor).

WO 2014/079850 discloses both substituted heterocyclic compounds which were believed to stimulate adult neuronal stem cells and that said compounds may be used for a plurality of different diseases. However, although neuronal stem cells have the ability to differentiate into several cell types, it cannot be predicted whether said new cell types can be stimulated by the same compounds. However, a significant number of compounds which stimulate neuronal stem cells have no or only a weak activity with regard to other cell types such as retinal precursor cells.

U.S. Pat. No. 6,117,675 discloses stem cells isolated from the retina of mammals and retinal cells differentiated from these stem cells and a method for obtaining cells from a retinal pigment epithelial layer of a mammal.

There is currently no way to reverse permanent damage to the retina and restore vision. Drug treatments focus on treating the illness and its symptoms to prevent further damage to the retina. There is a need to reverse damage to the retina and restore vision by endogenously generating new retinal cells or transplanting retinal cells.

The term “precursor cells” encompasses in this context any form of proliferative and non-proliferative cells such as stem cells per se and progenitor cells that can give rise to further differentiated functional tissues of the eye. Such precursor cells include in particular retinal precursor cells.

The problem of the present invention is therefore to provide new compounds, which stimulate the proliferation of retinal precursor cells.

The problem is solved by compounds of formula (I) and (Ia). Further preferred embodiments are subject of the dependent claims.

It has been shown that compounds of formula (I) and (Ia) stimulate production of mammalian retinal precursor cells. The selective activation of the endogenous precursor cells allows a controlled repair and regeneration of the retina. Thus, it is possible to restore vision by endogenously generating new precursor cells by a compound according to the present invention. Therefore, the compound is useful as a therapeutically active substance in the treatment of neuroretinal diseases, i.e. as a medicament.

Thus, the present invention relates to a compound of formula (I)

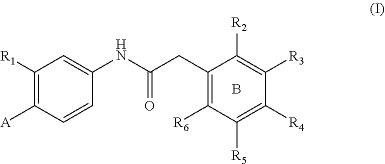

or a pharmaceutically acceptable salt thereof,
wherein:
A is a 5-oxazolyl residue or a pyridine-4-yl residue
R₁ is selected from the group consisting of fluoro and chloro;
R₂, R₃, R₄, R₅ and R₆ of the phenyl ring B are independently from each other selected from the group consisting of hydrogen, a linear or branched alkyl having 1 to 4 carbon atoms, trifluoromethyl, 2,2,2-trifluoroethyl, methylsulfanyl, ethylsulfanyl, methylsulfonyl, ethylsulfonyl, difluoromethoxy, trifluoromethoxy, fluoro, bromo, chloro, methoxy, ethoxy, propoxy, butoxy, hydroxy and amino; and
at least two of R₂, R₃, R₄, R₅ and R₆ are hydrogens, with the proviso that if R₁ is chloro, R₅ is not methoxy.

The term “pharmaceutically acceptable salt” stands for therapeutically active, non-toxic acid salt forms, which the compound according to the present invention is able to form.

The term “alkyl” as a group refers to a straight or branched hydrocarbon chain containing 1 to 4 of carbon atoms. Examples of “alkyl” as used herein include methyl, ethyl, n-propyl, isopropyl, n-butyl, isobutyl, sec-butyl and tert-butyl.

The residue A may be a 5-oxazolyl group of the formula (II)

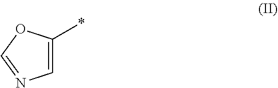 [SMILES: *c1cnco1]

wherein “*” denotes the point of attachment to the remainder of the molecule.

Alternatively, the residue A may be a pyridine group of the formula (III)

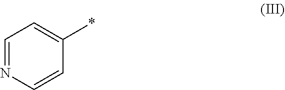 [SMILES: *c1ccncc1]

wherein “*” denotes the point of attachment to the remainder of the molecule.

Preferably, the phenyl ring B in the compound of the present invention is monosubstituted or disubstituted, but it is also possible that all of R₂, R₃, R₄, R₅ and R₆ are hydrogen. The term “monosubstituted” means that one of R₂, R₃, R₄, R₅ and R₆ is not hydrogen. The term “disubstituted” means that two of R₂, R₃, R₄, R₅ and R₆ are not hydrogens.

Preferably, in the compound of the present invention, R₁ is chloro. Said compounds show an outstanding biological activity.

In one embodiment of the present invention, R₁ is a residue as defined above and the phenyl ring B is not substituted, that is, all of R₂, R₃, R₄, R₅ and R₆ are hydrogens.

In another embodiment of the present invention, R₁ is a residue as defined above and the phenyl ring B is monosubstituted, that is, one of R₂, R₃, R₄, R₅ and R₆ is not hydrogen.

If the phenyl ring B is monosubstituted, R₂ is preferably selected from the group consisting of methyl, trifluoromethyl, methylsulfanyl, methylsulfonyl, difluoromethoxy, fluoro, bromo, chloro, methoxy, and ethoxy, most preferably difluoromethoxy and chloro, and R₂, R₃, R₄, R₅ and R₆ are hydrogen. Such a monosubstituted phenyl ring B with a bulky residue R₂ results in a particular good stimulation of retinal precursor cells.

Alternatively, if the phenyl ring B is monosubstituted, R₂ is hydrogen and one of R₃, R₄, R₅ and R₆ is preferably selected from the group consisting of trifluoromethyl, difluoromethoxy, methoxy, preferably trifluoromethyl and difluoromethoxy.

In another embodiment of the present invention, the phenyl ring B is disubstituted, that is, two of R₂, R₃, R₄, R₅ and R₆ are not hydrogens. The disubstitution may be an ortho, meta or para substitution.

Preferably, R₂ is selected from the group consisting of fluoro, bromo and chloro, and one of R₃, R₄ or R₅ is selected from the group consisting of fluoro, bromo and chloro. The two residues which are different from hydrogen may be the same or different from each other. Preferably, R₂ is chloro and R₅ is fluoro resulting in a para-substitution, or both R₂ and R₄ are fluoro resulting in a meta-substitution.

Preferably, the compound of formula (I) is selected from the group consisting of compounds of the formula (I), wherein A, R₁, R₂, R₃, R₄, R₅ and R₆ are

TABLE 1

| A | R₁ | R₂ | R₃ | R₄ | R₅ | R₆ |
|---|---|---|---|---|---|---|
| 5-oxazolyl | Cl | Cl | H | H | H | H |
| 5-oxazolyl | Cl | H | Cl | H | H | H |
| 5-oxazolyl | Cl | H | H | Cl | H | H |
| 5-oxazolyl | Cl | H | H | H | Cl | H |
| 5-oxazolyl | Cl | F | H | H | H | H |
| 5-oxazolyl | Cl | H | F | H | H | H |
| 5-oxazolyl | Cl | H | H | F | H | H |
| 5-oxazolyl | Cl | H | H | H | F | H |
| 5-oxazolyl | Cl | Br | H | H | H | H |
| 5-oxazolyl | Cl | H | Br | H | H | H |
| 5-oxazolyl | Cl | H | H | Br | H | H |
| 5-oxazolyl | Cl | H | H | H | Br | H |
| 5-oxazolyl | Cl | CF₃ | H | H | H | H |
| 5-oxazolyl | Cl | H | CF₃ | H | H | H |
| 5-oxazolyl | Cl | H | H | CF₃ | H | H |
| 5-oxazolyl | Cl | H | H | H | CF₃ | H |
| 5-oxazolyl | Cl | OCH₃ | H | H | H | H |
| 5-oxazolyl | Cl | H | OCH₃ | H | H | H |
| 5-oxazolyl | Cl | H | H | OCH₃ | H | H |
| 5-oxazolyl | Cl | H | H | H | OCH₃ | H |
| 5-oxazolyl | Cl | CH₃ | H | H | H | H |
| 5-oxazolyl | Cl | H | CH₃ | H | H | H |
| 5-oxazolyl | Cl | H | H | CH₃ | H | H |
| 5-oxazolyl | Cl | H | H | H | CH₃ | H |
| 5-oxazolyl | Cl | OCHF₂ | H | H | H | H |
| 5-oxazolyl | Cl | H | OCHF₂ | H | H | H |
| 5-oxazolyl | Cl | H | H | OCHF₂ | H | H |
| 5-oxazolyl | Cl | H | H | H | OCHF₂ | H |
| 5-oxazolyl | Cl | SO₂CH₃ | H | H | H | H |
| 5-oxazolyl | Cl | H | SO₂CH₃ | H | H | H |
| 5-oxazolyl | Cl | H | H | SO₂CH₃ | H | H |
| 5-oxazolyl | Cl | H | H | H | SO₂CH₃ | H |
| 5-oxazolyl | F | Cl | H | H | H | H |
| 5-oxazolyl | F | H | Cl | H | H | H |
| 5-oxazolyl | F | H | H | Cl | H | H |
| 5-oxazolyl | F | H | H | H | Cl | H |
| 5-oxazolyl | F | F | H | H | H | H |
| 5-oxazolyl | F | H | F | H | H | H |
| 5-oxazolyl | F | H | H | F | H | H |
| 5-oxazolyl | F | H | H | H | F | H |
| 5-oxazolyl | F | Br | H | H | H | H |
| 5-oxazolyl | F | H | Br | H | H | H |
| 5-oxazolyl | F | H | H | Br | H | H |
| 5-oxazolyl | F | H | H | H | Br | H |
| 5-oxazolyl | F | CF₃ | H | H | H | H |
| 5-oxazolyl | F | H | CF₃ | H | H | H |
| 5-oxazolyl | F | H | H | CF₃ | H | H |
| 5-oxazolyl | F | H | H | H | CF₃ | H |
| 5-oxazolyl | F | OCH₃ | H | H | H | H |
| 5-oxazolyl | F | H | OCH₃ | H | H | H |
| 5-oxazolyl | F | H | H | OCH₃ | H | H |
| 5-oxazolyl | F | H | H | H | OCH₃ | H |
| 5-oxazolyl | F | CH₃ | H | H | H | H |
| 5-oxazolyl | F | H | CH₃ | H | H | H |
| 5-oxazolyl | F | H | H | CH₃ | H | H |
| 5-oxazolyl | F | H | H | H | CH₃ | H |
| 5-oxazolyl | F | OCHF₂ | H | H | H | H |
| 5-oxazolyl | F | H | OCHF₂ | H | H | H |
| 5-oxazolyl | F | H | H | OCHF₂ | H | H |
| 5-oxazolyl | F | H | H | H | OCHF₂ | H |
| 5-oxazolyl | F | SO₂CH₃ | H | H | H | H |
| 5-oxazolyl | F | H | SO₂CH₃ | H | H | H |
| 5-oxazolyl | F | H | H | SO₂CH₃ | H | H |
| 5-oxazolyl | F | H | H | H | SO₂CH₃ | H |
| 5-oxazolyl | F | F | H | F | H | H |
| 5-oxazolyl | F | F | H | H | F | H |
| 5-oxazolyl | F | F | H | Cl | H | H |
| 5-oxazolyl | F | F | H | H | Cl | H |
| 5-oxazolyl | Cl | F | H | F | H | H |
| 5-oxazolyl | Cl | F | H | H | F | H |
| 5-oxazolyl | Cl | F | H | Cl | H | H |
| 5-oxazolyl | Cl | F | H | H | Cl | H |
| pyridine-4-yl | Cl | Cl | H | H | H | H |
| pyridine-4-yl | Cl | H | Cl | H | H | H |
| pyridine-4-yl | Cl | H | H | Cl | H | H |
| pyridine-4-yl | Cl | H | H | H | Cl | H |
| pyridine-4-yl | Cl | F | H | H | H | H |
| pyridine-4-yl | Cl | H | F | H | H | H |
| pyridine-4-yl | Cl | H | H | F | H | H |
| pyridine-4-yl | Cl | H | H | H | F | H |
| pyridine-4-yl | Cl | Br | H | H | H | H |
| pyridine-4-yl | Cl | H | Br | H | H | H |
| pyridine-4-yl | Cl | H | H | Br | H | H |
| pyridine-4-yl | Cl | H | H | H | Br | H |
| pyridine-4-yl | Cl | CF₃ | H | H | H | H |
| pyridine-4-yl | Cl | H | CF₃ | H | H | H |
| pyridine-4-yl | Cl | H | H | CF₃ | H | H |
| pyridine-4-yl | Cl | H | H | H | CF₃ | H |
| pyridine-4-yl | Cl | OCH₃ | H | H | H | H |
| pyridine-4-yl | Cl | H | OCH₃ | H | H | H |
| pyridine-4-yl | Cl | H | H | OCH₃ | H | H |
| pyridine-4-yl | Cl | CH₃ | H | H | H | H |
| pyridine-4-yl | Cl | H | CH₃ | H | H | H |
| pyridine-4-yl | Cl | H | H | CH₃ | H | H |
| pyridine-4-yl | Cl | H | H | H | CH₃ | H |
| pyridine-4-yl | Cl | OCHF₂ | H | H | H | H |
| pyridine-4-yl | Cl | H | OCHF₂ | H | H | H |
| pyridine-4-yl | Cl | H | H | OCHF₂ | H | H |
| pyridine-4-yl | Cl | H | H | H | OCHF₂ | H |
| pyridine-4-yl | Cl | SO₂CH₃ | H | H | H | H |
| pyridine-4-yl | Cl | H | SO₂CH₃ | H | H | H |
| pyridine-4-yl | Cl | H | H | SO₂CH₃ | H | H |
| pyridine-4-yl | Cl | H | H | H | SO₂CH₃ | H |
| pyridine-4-yl | F | Cl | H | H | H | H |
| pyridine-4-yl | F | H | Cl | H | H | H |
| pyridine-4-yl | F | H | H | Cl | H | H |
| pyridine-4-yl | F | H | H | H | Cl | H |
| pyridine-4-yl | F | F | H | H | H | H |
| pyridine-4-yl | F | H | F | H | H | H |
| pyridine-4-yl | F | H | H | F | H | H |
| pyridine-4-yl | F | H | H | H | F | H |
| pyridine-4-yl | F | Br | H | H | H | H |
| pyridine-4-yl | F | H | Br | H | H | H |
| pyridine-4-yl | F | H | H | Br | H | H |
| pyridine-4-yl | F | H | H | H | Br | H |
| pyridine-4-yl | F | CF₃ | H | H | H | H |
| pyridine-4-yl | F | H | CF₃ | H | H | H |
| pyridine-4-yl | F | H | H | CF₃ | H | H |
| pyridine-4-yl | F | H | H | H | CF₃ | H |
| pyridine-4-yl | F | OCH₃ | H | H | H | H |
| pyridine-4-yl | F | H | OCH₃ | H | H | H |
| pyridine-4-yl | F | H | H | OCH₃ | H | H |
| pyridine-4-yl | F | H | H | H | OCH₃ | H |
| pyridine-4-yl | F | CH₃ | H | H | H | H |
| pyridine-4-yl | F | H | CH₃ | H | H | H |
| pyridine-4-yl | F | H | H | CH₃ | H | H |
| pyridine-4-yl | F | H | H | H | CH₃ | H |
| pyridine-4-yl | F | OCHF₂ | H | H | H | H |
| pyridine-4-yl | F | H | OCHF₂ | H | H | H |
| pyridine-4-yl | F | H | H | OCHF₂ | H | H |
| pyridine-4-yl | F | H | H | H | OCHF₂ | H |
| pyridine-4-yl | F | SO₂CH₃ | H | H | H | H |
| pyridine-4-yl | F | H | SO₂CH₃ | H | H | H |
| pyridine-4-yl | F | H | H | SO₂CH₃ | H | H |
| pyridine-4-yl | F | H | H | H | SO₂CH₃ | H |
| pyridine-4-yl | F | F | H | F | H | H |
| pyridine-4-yl | F | F | H | H | F | H |
| pyridine-4-yl | F | F | H | Cl | H | H |
| pyridine-4-yl | F | F | H | H | Cl | H |
| pyridine-4-yl | Cl | F | H | F | H | H |
| pyridine-4-yl | Cl | F | H | H | F | H |
| pyridine-4-yl | Cl | F | H | Cl | H | H |
| pyridine-4-yl | Cl | F | H | H | Cl | H |

Especially good results could be obtained by the following compounds according to the present invention:

TABLE 2

| | | Cell |
|---|---|---|
| | | proliferation |
| Comp. | | within one week |
| No. | Chemical structure | [%] |
| 1 | 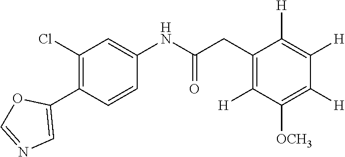 [SMILES: COc1cccc(CC(=O)Nc2ccc(-c3cnco3)c(Cl)c2)c1] | 203 |
| 2 | 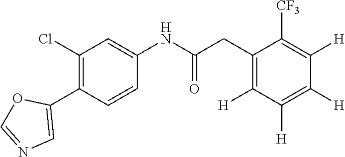 [SMILES: O=C(Cc1ccccc1C(F)(F)F)Nc1ccc(-c2cnco2)c(Cl)c1] | 142 |
| 3 | 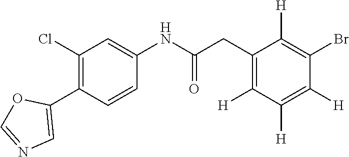 [SMILES: O=C(Cc1cccc(Br)c1)Nc1ccc(-c2cnco2)c(Cl)c1] | 143 |
| 4 | 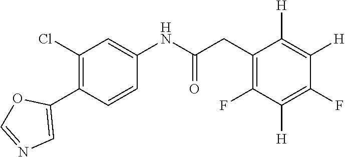 [SMILES: O=C(Cc1ccc(F)cc1F)Nc1ccc(-c2cnco2)c(Cl)c1] | 157 |
| 5 | 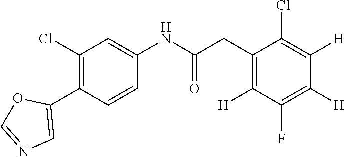 [SMILES: O=C(Cc1cc(F)ccc1Cl)Nc1ccc(-c2cnco2)c(Cl)c1] | 121 |
| 6 | 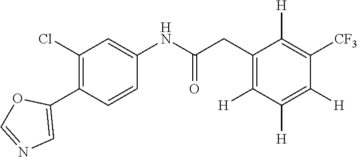 [SMILES: O=C(Cc1cccc(C(F)(F)F)c1)Nc1ccc(-c2cnco2)c(Cl)c1] | 134 |
| 7 | 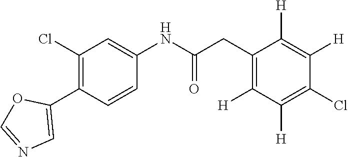 [SMILES: O=C(Cc1ccc(Cl)cc1)Nc1ccc(-c2cnco2)c(Cl)c1] | 133 |
| 8 | 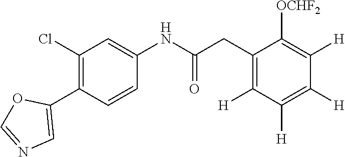 [SMILES: O=C(Cc1ccccc1OC(F)F)Nc1ccc(-c2cnco2)c(Cl)c1] | 135 |
| 9 | 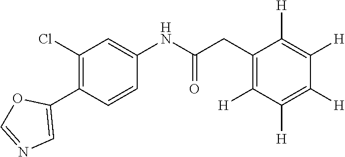 [SMILES: O=C(Cc1ccccc1)Nc1ccc(-c2cnco2)c(Cl)c1] | 149 |
| 10 | 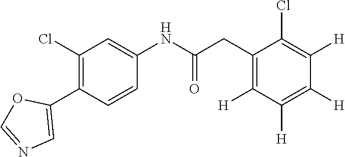 [SMILES: O=C(Cc1ccccc1Cl)Nc1ccc(-c2cnco2)c(Cl)c1] | 158 |
| 11 | 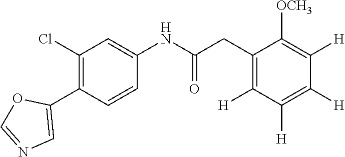 [SMILES: COc1ccccc1CC(=O)Nc1ccc(-c2cnco2)c(Cl)c1] | 128 |
| 12 | 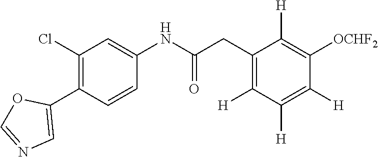 [SMILES: O=C(Cc1cccc(OC(F)F)c1)Nc1ccc(-c2cnco2)c(Cl)c1] | 122 |
| 13 | 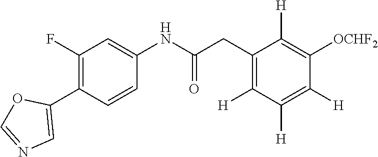 [SMILES: O=C(Cc1cccc(OC(F)F)c1)Nc1ccc(-c2cnco2)c(F)c1] | 120 |
| 14 | 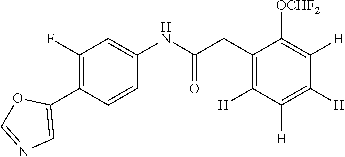 [SMILES: O=C(Cc1ccccc1OC(F)F)Nc1ccc(-c2cnco2)c(F)c1] | 119 |
| 15 | 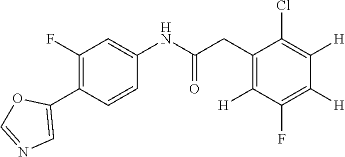 [SMILES: O=C(Cc1cc(F)ccc1Cl)Nc1ccc(-c2cnco2)c(F)c1] | 133 |
| 16 | 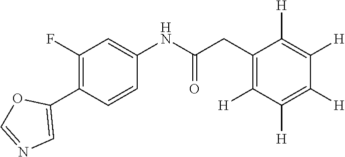 [SMILES: O=C(Cc1ccccc1)Nc1ccc(-c2cnco2)c(F)c1] | 123 |
| 17 | 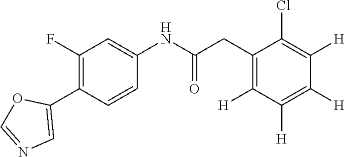 [SMILES: O=C(Cc1ccccc1Cl)Nc1ccc(-c2cnco2)c(F)c1] | 137 |
| 18 | 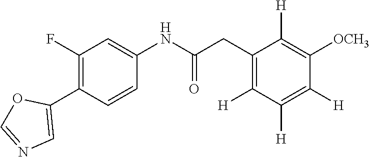 [SMILES: COc1cccc(CC(=O)Nc2ccc(-c3cnco3)c(F)c2)c1] | 127 |
| 19 | 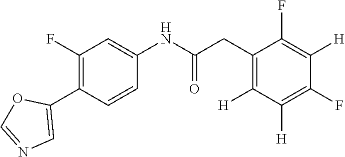 [SMILES: O=C(Cc1ccc(F)cc1F)Nc1ccc(-c2cnco2)c(F)c1] | 140 |
| 20 | 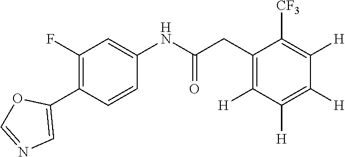 [SMILES: O=C(Cc1ccccc1C(F)(F)F)Nc1ccc(-c2cnco2)c(F)c1] | 118 |
| C* | — | 100 |

C* = Control experiment (absence of a compound according to the present invention)

In particular, the compounds of formula (1), (10), (4), (9), (3) and (2) show excellent results with regard to the stimulation of precursor cells, and in particular of retinal precursor cells. Within one week, the compound of formula (1) showed an increase of cell proliferation of 103%, the compound of formula (10) of 58%, compound of formula (4) of 57%, compound of formula (9) of 49%, compound of formula (3) of 43%, and compound of formula (2) of 42%.

In a further embodiment, the present invention relates to a pharmaceutical composition comprising a pharmaceutically acceptable carrier and/or adjuvant; and a compound of the formula (Ia)

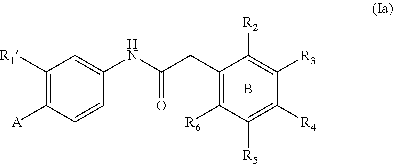

or a pharmaceutically acceptable salt thereof, wherein
A is a 5-oxazolyl residue or a pyridine-4-yl residue
R₁′ is selected from the group consisting of methoxy, hydrogen, fluoro and chloro;
R₂, R₃, R₄, R₅ and R₆ of the phenyl ring B are independently from each other selected from the group consisting of hydrogen, a linear or branched alkyl having 1 to 4 carbon atoms, trifluoromethyl, 2,2,2-trifluoroethyl, methylsulfanyl, ethylsulfanyl, methylsulfonyl, ethylsulfonyl, difluoromethoxy, trifluoromethoxy, fluoro, bromo, chloro, methoxy, ethoxy, propoxy, butoxy, hydroxy and amino; and
at least two of R₂, R₃, R₄, R₅ and R₆ are hydrogens,
with the proviso that if R₁′ is hydrogen or methoxy, A is a pyridine-4-yl residue.
as a therapeutically active substance.

The definition of the compound of formula (Ia) differs from the definition of the compound of formula (I) in that R₁′ is selected from the group consisting of methoxy, hydrogen, fluoro and chloro instead of R₁ that was only selected from the group consisting of fluoro and chloro.

The term “prevention” refers to the prevention or reduction of signs and symptoms associated with neuroretinal diseases, in particular of primary neuroretinal diseases leading to photoreceptor loss or degeneration of the photoreceptor layer of the retina in subjects who are at risk for developing the disease. In these subjects a predisposing factor may be retained, but the signs and/or symptoms of the disease do not occur or take significantly longer to develop. Further, it also includes the prevention of a further deterioration of the symptoms once the disease has occurred.

The term “pharmaceutical composition” as used here means a composition that is suitable for administering to human patients for the treatment of diseases. Said pharmaceutical composition efficiently stimulates proliferation, migration or both proliferation and migration of endogenous retinal precursor cells in a patient.

In a preferred embodiment of the present invention, the pharmaceutical composition comprises a pharmaceutically acceptable carrier and/or adjuvant; and a compound of the formula (I) as defined above, and in particular a compound of formula (I) as disclosed in Table 1 and/or Table 2.

In another embodiment of the present invention, the pharmaceutical composition comprises the compound of formula (Ia), wherein R₁, R₂, R₃, R₄, R₅ and R₆ are

TABLE 3

| A | R₁ | R₂ | R₃ | R₄ | R₅ | R₆ |
|---|---|---|---|---|---|---|
| pyridine-4-yl | OCH₃ | Cl | H | H | H | H |
| pyridine-4-yl | OCH₃ | H | Cl | H | H | H |
| pyridine-4-yl | OCH₃ | H | H | Cl | H | H |
| pyridine-4-yl | OCH₃ | H | H | H | Cl | H |
| pyridine-4-yl | OCH₃ | F | H | H | H | H |
| pyridine-4-yl | OCH₃ | H | F | H | H | H |
| pyridine-4-yl | OCH₃ | H | H | F | H | H |
| pyridine-4-yl | OCH₃ | H | H | H | F | H |
| pyridine-4-yl | OCH₃ | Br | H | H | H | H |
| pyridine-4-yl | OCH₃ | H | Br | H | H | H |
| pyridine-4-yl | OCH₃ | H | H | Br | H | H |
| pyridine-4-yl | OCH₃ | H | H | H | Br | H |
| pyridine-4-yl | OCH₃ | CF₃ | H | H | H | H |
| pyridine-4-yl | OCH₃ | H | CF₃ | H | H | H |
| pyridine-4-yl | OCH₃ | H | H | CF₃ | H | H |
| pyridine-4-yl | OCH₃ | H | H | H | CF₃ | H |
| pyridine-4-yl | OCH₃ | OCH₃ | H | H | H | H |
| pyridine-4-yl | OCH₃ | H | OCH₃ | H | H | H |
| pyridine-4-yl | OCH₃ | H | H | OCH₃ | H | H |
| pyridine-4-yl | OCH₃ | H | H | H | OCH₃ | H |
| pyridine-4-yl | OCH₃ | CH₃ | H | H | H | H |
| pyridine-4-yl | OCH₃ | H | CH₃ | H | H | H |
| pyridine-4-yl | OCH₃ | H | H | CH₃ | H | H |
| pyridine-4-yl | OCH₃ | H | H | H | CH₃ | H |
| pyridine-4-yl | OCH₃ | OCHF₂ | H | H | H | H |
| pyridine-4-yl | OCH₃ | H | OCHF₂ | H | H | H |
| pyridine-4-yl | OCH₃ | H | H | OCHF₂ | H | H |
| pyridine-4-yl | OCH₃ | H | H | H | OCHF₂ | H |
| pyridine-4-yl | OCH₃ | SO₂CH₃ | H | H | H | H |
| pyridine-4-yl | OCH₃ | H | SO₂CH₃ | H | H | H |
| pyridine-4-yl | OCH₃ | H | H | SO₂CH₃ | H | H |
| pyridine-4-yl | OCH₃ | H | H | H | SO₂CH₃ | H |
| pyridine-4-yl | OCH₃ | F | H | F | H | H |
| pyridine-4-yl | OCH₃ | F | H | H | F | H |
| pyridine-4-yl | OCH₃ | F | H | Cl | H | H |
| pyridine-4-yl | OCH₃ | F | H | H | Cl | H |

Especially good results could be obtained by the following compounds according to the present invention:

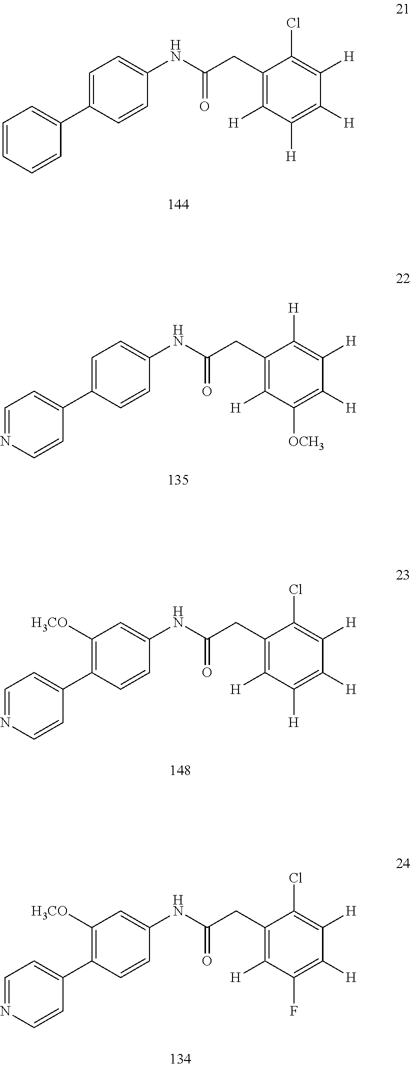 [SMILES: O=C(Cc1ccccc1Cl)Nc1ccc(-c2ccccc2)cc1 ; COc1cccc(CC(=O)Nc2ccc(-c3ccncc3)cc2)c1 ; COc1cc(NC(=O)Cc2ccccc2Cl)ccc1-c1ccncc1 ; COc1cc(NC(=O)Cc2cc(F)ccc2Cl)ccc1-c1ccncc1]

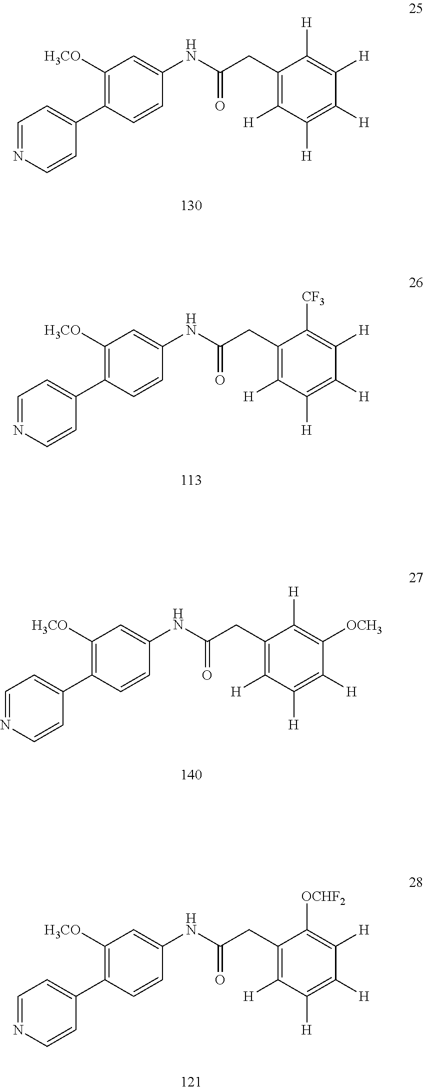 [SMILES: COc1cc(NC(=O)Cc2ccccc2)ccc1-c1ccncc1 ; COc1cc(NC(=O)Cc2ccccc2C(F)(F)F)ccc1-c1ccncc1 ; COc1cccc(CC(=O)Nc2ccc(-c3ccncc3)c(OC)c2)c1 ; COc1cc(NC(=O)Cc2ccccc2OC(F)F)ccc1-c1ccncc1]

In particular, the compounds of formula (23), (21) (22), (24) and (27) show excellent results with regard to the stimulation of precursor cells, and in particular of retinal precursor cells. Within one week, the compound of formula (23) showed an increase of cell proliferation of 48%, the compound of formula (21) of 44%, the compound of formula (22) of 35%, the compound of formula (24) of 34%, and compound of formula (27) of 40%.

As already mentioned, it could be shown that the compounds according to the present invention and the compositions according to the present invention stimulate the proliferation of retinal precursor cells. Thus, they are suitable in the treatment and/or prevention of neuroretinal diseases, in particular of primary neuroretinal diseases leading to photoreceptor loss or degeneration of the photoreceptor layer of the retina.

Compounds and compositions according to the present invention are suitable for the use in the treatment and/or prevention of a disease selected from the group consisting of inherited retinal dystrophies including retinitis pigmentosa (RP), including syndromic and non-syndromic forms, X-chromosome linked, recessive, dominant and sporadic forms, rod-cone dystrophies, Usher's syndrome, Stargardt's disease, cone-rod dystrophies, cone dystrophies, achromatopsia, blue cone monochromacy, enhanced S-cone syndrome, rod dystrophies, choroideremia, Leber's congenital amaurosis, juvenile X-chromosome linked retinoschisis (JXLR), fundus albipunctatus, retinitis punctata albescens, fleck retina of Kandori, bietti crystalline retinal dystrophy, fenestrated sheen macular dystrophy, adult-onset foveomacular vitelliform dystrophy, Batten's disease, congenital stationary night blindness, familial exudative vitreoretinopathy (FEVR), ocular albinism, oculocutaneous albinism, fovea hypoplasia, abetalipoproteinemia, Stickler syndrome and retinal dystrophy (Bothnia type). Most preferably, the compound of the present invention is used in the treatment of retinitis pigmentosa (RP), including syndromic and non-syndromic forms, X-chromosome linked, recessive, dominant and sporadic forms.

Compounds and compositions according to the present invention are suitable for the use in the treatment and/or prevention of acquired degeneration selected from the group consisting of crystalline maculopathy (drug-related, hyperoxaluria, cystinosis, Sjogren-Larsson syndrome), west African crystalline maculopathy, solar retinopathy, talc retinopathy, diabetic retinopathy, sickle cell retinopathy, macular telangectasia, eales disease, retinal detachment, retinal dialysis, peripheral retinoschisis.

Compounds and compositions according to the present invention are suitable for the use in the treatment and/or prevention of vascular related retinal degeneration selected from the group consisting of central/branch retinal artery occlusion (CRAO/BRAO), central/branch retinal vein occlusion (CRVO/BRVO), haemorrhagic occlusive retinal vasculitis (HORV).

Compounds and compositions according to the present invention are suitable for the use in the treatment and/or prevention of drug-induced maculopathies selected from the group consisting of chloroquine, hydroxychloroquine, phenothiazine, quinine sulfate, thioridazine, clofazimine, cholopromazine, deferoxamine, chloroquine-derivatives, cisplatin, carmustine, chlofazimine and vigabatrin as well as crystal-induced maculopathies including tamoxifen, talc, canthaxanthine, methoxyflurane, nitrofurantoin, cystoid macular edema (CME) including Epinephrine, latanoprost and nicotinic acid.

Compounds and compositions according to the present invention are suitable for the use in the treatment and/or prevention of infectious and/or inflammatory eye diseases selected from the group consisting of progressive outer retinal necrosis (PORN), acute retinal necrosis (ARN), CMV-retinitis, Sarcoidosis, acute syphilitic posterior placoid chorioretinitis, tuberculosis chorioretinitis, toxoplasmic retinochoroiditis, posterior Uveitis and retinal vasculitis, intermediate uveitis, pars planitis +/− CME, enophthalmitis (anterior and/or posterior), posterior scleritis and masquerade syndromes.

Compounds and compositions according to the present invention are suitable for the use in the treatment and/or prevention of white dot syndromes selected from the group consisting of multifocal choroiditis and panuveitis (MCP), punctate inner choroidopathy (PIC), birdshot retinochoroidopathy, acute macular neuroretinopathy (AMN) and acute zonal occult outer retinopathy (AZOOR).

The compound or the composition according to the present invention can be administered to a patient, either alone or in combination with one or more additional therapeutic agents. “Patient” as used herein, includes mammals such as humans, non-human primates, rats, mice, rabbits, hares, dogs, cats, horses, cows and pigs, preferably human.

The pharmaceutical composition according to the present invention may comprise one or more additional therapeutic agents.

Preferably, such a pharmaceutical composition provides controlled release properties. The term “controlled release pharmaceutical compositions” herein refers to any composition or dosage form, which comprises the compound of the present invention and which is formulated to provide a longer duration of pharmacological response after administration of the dosage form than is ordinarily experienced after administration of a corresponding immediate release composition comprising the same drug in the same amount. Controlled release may be extended up to several months depending on the matrix used. Preferably, the release of the compound according to the present invention takes place over a period of up to 12 months, most preferably over a period of up to 6 months. Such a controlled release formulation results in an increased patient comfort and in significant lower costs.

The matrix material used for a pharmaceutical composition according to the present may comprise hydrophobic release controlling agents. It is preferably selected from but not limited to polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, carnauba wax, paraffin wax, microcrystalline wax, and ozokerite; fatty alcohols such as cetostearyl alcohol, stearyl alcohol, cetyl alcohol and myristyl alcohol, and fatty acid esters such as glyceryl monostearate; glycerol monooleate, acetylated monoglycerides, tristearin, tripalmitin, cetyl esters wax, glyceryl palmitostearate, glyceryl behenate, or hydrogenated vegetable oils.

The compound of the invention can be delivered to the eye through a variety of routes, including but not limited to topical application to the eye or by intraocular injection into, for example, the vitreous or subretinal (interphotoreceptor) space; locally by insertion or injection into the tissue surrounding the eye; systemically through an oral route or by subcutaneous, intravenous or intramuscular injection; or via catheter or implant. Most preferably, the compound of the present invention is delivered by intraocular injection. The compound of the invention can be administered prior to the onset of the condition to prevent its occurrence, such as during eye surgery, immediately after the onset of the pathological condition, or during the occurrence of an acute or protracted condition.

Depending on the intended mode of administration, the compound according to the present invention may be incorporated in any pharmaceutically acceptable dosage form, such as for example, liquids, including solutions, suspensions and emulsions, tablets, suppositories, pills, capsules, powders or the like, preferably dosage forms suitable for single administration of precise dosages, or sustained release dosage forms for continuous controlled administration. Most preferred are liquids.

Liquid pharmaceutically administrable dosage forms can be for example a solution, a suspension or an emulsion, preferably a solution comprising a compound of the present invention and optional pharmaceutical adjutants in a carrier, such as for example, water, saline, aqueous dextrose, glycerol, ethanol, DMSO and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate and triethanolamine oleate.

The present invention also relates to a method of treating a neuroretinal disease that leads to photoreceptor loss or outer-retina degeneration, comprising administering a compound of formula (Ia) or a pharmaceutically acceptable salt thereof to a patient having the retinal disease so as to be delivered to an eye of the patient in an amount effective to treat the retinal disease. The compound of formula (Ia) is defined above in detail.

### Preparation of the Compounds of the Invention

The compounds of formula (I) may be prepared by methods described below, together with synthetic methods known in the art of organic chemistry, or modifications that are familiar to those of ordinary skill in the art. The starting materials used herein are available commercially or may be prepared by routine methods known in the art, such as those methods described in standard reference books such as “Compendium of Organic Synthetic Methods, Vol. I-XlN” (published with Wiley-Interscience, ISSN: 1934-4783). Preferred methods include, but are not limited to, those described below.

The schemes are representative of methods useful in synthesizing the compounds of the present invention and the supporting examples. They are not to constrain the scope of the invention in anyway.

### Preparative HPLC

Preparative high-pressure liquid chromatography (HPLC) used to purify reaction mass in the following examples and preparations was effected according to the following method unless modified in specific examples. A Waters auto purification instrument with a YMC Triart C18 (250×21.2 mm, 5p) column operated at rt with a flow rate of 16 mL/min. Samples were eluted with 20 mM ammonium bicarbonate in water (mobile phase A) and acetonitrile (mobile phase B) and a gradient profile of 70% A and 30% B initially, then 45% A and 55% B in 3 min, adapted to 20% A and 80% B in 20 min, then to 5% A and 95% B in 21 min, which was held constant for 2 min. Pure fractions were concentrated to yield the final product.

### Analytical HPLC

Analytical ultra-performance liquid chromatography (UPLC) used in the following examples and preparations was effected according to the following method unless modified in specific examples. A Chromegabond WR C18 (3 cm×3.2 mm, 3p) column operated with a flow rate of 1.5 mL/min. As mobile phases, 0.02% TFA in water (mobile phase C) and 0.02% TFA in CH₃CN (mobile phase D) were used in a gradient starting at 90% C and 10% D, changed to 10% C and 90% D in 3.0 min, then to 90% C and 10% D in 4.0 min, which was held constant up to 5.1 min.

### GENERAL METHODS—SYNTHESIS

### Method 1

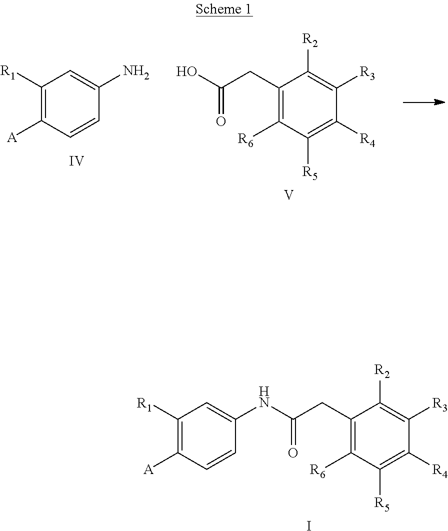

where R1, R2, R3, R4, R5, R6 are as described in formula I.

Compounds of general formula I (Scheme 1) may be prepared by reacting compounds of general formula IV with a carboxylic acid of general formula V using procedures known to chemists skilled in the art.

### Method 2

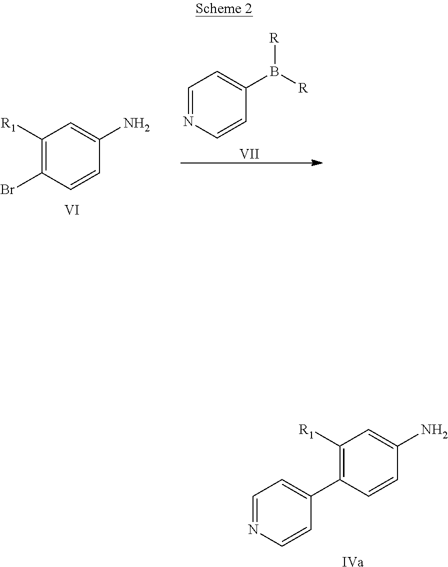

where R1 is as described in formula I, R are hydroxy groups or R together with the boron atom form a 4,4,5,5-tetramethyl-1,3,2-dioxaborolane group.

Compounds of general formula IVa (Scheme 2) may be prepared from compounds of general formulae VI and VII in the presence of a palladium catalyst such as tetrakis(triphenylphosphin)palladium(0) and a base such as potassium carbonate or other Suzuki-Miyaura coupling reaction conditions known to chemists skilled in the art of organic synthesis.

### Method 3

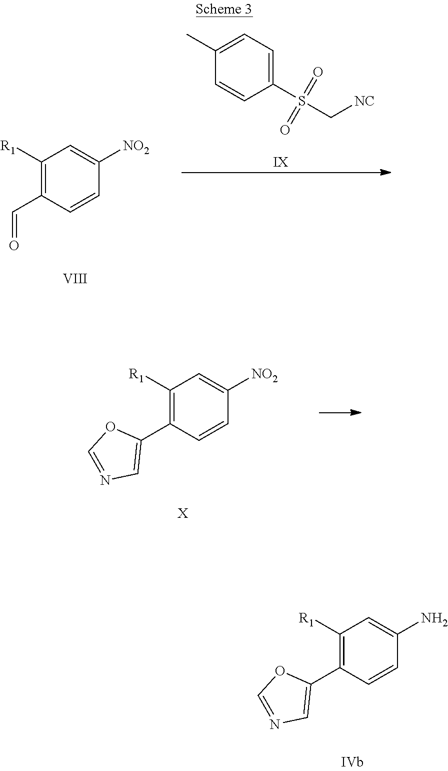

where R1 is as described in formula I.

Compounds of general formula IVb (Scheme 3) may be prepared by reduction of the nitro group in compounds of general formula X using procedures known to chemists skilled in the art. Compounds of general formula X may be prepared from aldehydes of general formula VIII by reaction in the presence of a reagent such as isocyanomethane)sulfonyl-4-methylbenzene (IX) in the presence of a base such as potassium carbonate.

### Synthesis of compounds comprising a 5-oxazolyl residue

### Intermediate 1

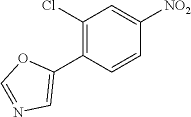 [SMILES: O=[N+]([O-])c1ccc(-c2cnco2)c(Cl)c1]

### 5-(2-chloro-4-nitrophenyl)oxazole

To a stirred solution of 2-chloro-4-nitrobenzaldehyde (3.00 g, 16.2 mmol) in methanol (20 mL) was added 1-(isocyanomethane)sulfonyl-4-methylbenzene (3.80 g, 19.5 mmol) followed by K₂CO₃ (8.00 g, 58.0 mmol) and the reaction mixture was heated to 80° C. and let cool down to rt over 2 h. After completion of the reaction, reaction mass was poured into sat NaHCO₃ solution (20 mL) and extracted into ethyl acetate (3×200 mL). The organic layer was washed with water, brine, dried over anhydrous sodium sulphate and concentrated under vacuum to get a crude which was purified by column chromatography using silica (100-200) (eluted at 30% ethyl acetate in hexane) to get 5-(2-chloro-4-nitrophenyl)1,3-oxazole (Intermediate 1) (2.9 g, 80%) as yellow solid. LCMS: 225.2 (M+H).

### Intermediate 2

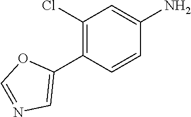 [SMILES: Nc1ccc(-c2cnco2)c(Cl)c1]

### 3-chloro-4-(1,3-oxazol-5-yl)aniline

To a stirred solution of 5-(2-chloro-4-nitrophenyl)-1,3-oxazole (Intermediate 1) (3 g, 13.39 mmol) in EtOH (40 mL) were added SnCl2 dihydrate (12.08 g, 53.57 mmol) and conc. HCl (5 mL) dropwise at 0° C. and the reaction mixture was stirred for 30 min at 80° C. After completion of the reaction, the reaction mass was neutralized by 2N NaOH solution and extracted with ethyl acetate (2×50 mL). The organic layer was thoroughly washed with water, dried over anhydrous sodium sulphate and concentrated under vacuum to afford 3-chloro-4-(1,3-oxazol-5-yl)aniline (Intermediate 2) (1.5 g, 57%) as yellow solid. LCMS: 195 (M+H).

### Intermediate 3

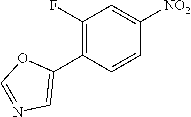 [SMILES: O=[N+]([O-])c1ccc(-c2cnco2)c(F)c1]

### 5-(2-fluoro-4-nitrophenyl)oxazole

To a stirred solution of 2-fluoro-4-nitro benzaldehyde (5 g, 29.56 mmol) and 1-(isocyanomethane)sulfonyl-4-methylbenzene (7.5 g, 38.43 mmol) in MeOH (35 mL) was added K₂CO₃ (16.3 g, 118.27 mmol) and the reaction mixture was heated to 80° C. for 2 h. After completion of the reaction, reaction mass was poured into saturated NaHCO₃ solution (50 mL) and extracted with ethyl acetate (2×50 mL). The organic layer was washed with water, brine, dried over anhydrous sodium sulphate and concentrated under vacuum to get a crude which was purified by column chromatography using silica (100-200) (eluted with 30% ethyl acetate in hexane) to afford 5-(2-fluoro-4-nitrophenyl)-1,3-oxazole (Intermediate 3) (2.5 g, 40%) as yellow solid. LCMS: 209.2 (M+H).

### Intermediate 4

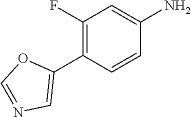 [SMILES: Nc1ccc(-c2cnco2)c(F)c1]

### 3-fluoro-4-(oxazole-5-yl)aniline

To a stirred solution of 5-(2-fluoro-4-nitrophenyl)-1,3-oxazole (Intermediate 3) (700 mg, 3.36 mmol) in EtOH (35 mL) were added tin(II) chloride SnCl2 dihydrate (3.03 g, 13.46 mmol) and conc. HCl (2 mL) dropwise at 0° C. and the reaction mixture was stirred for 30 min at 80° C. After completion of the reaction, the reaction mass was neutralized with a 2N NaOH solution and extracted with ethyl acetate (2×50 mL). The organic layer was thoroughly washed with water, dried over anhydrous sodium sulphate and concentrated under vacuum to afford 3-fluoro-4-(1,3-oxazol-5-yl)aniline (Intermediate 4) (350 mg, 53%) as yellow solid. LCMS: 179 (M+H).

### Synthesis of Compound (1)

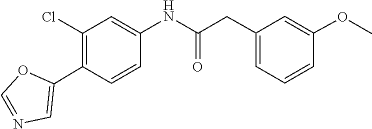 [SMILES: COc1cccc(CC(=O)Nc2ccc(-c3cnco3)c(Cl)c2)c1]

### N-(3-chloro-4-(oxazol-5-yl)phenyl)-2-(3-methoxyphenyl)acetamide

To a stirred solution of 3-chloro-4-(oxazole-5-yl)aniline (Intermediate 2) (100 mg, 0.51 mmol) and 2-(3-methoxyphenyl)acetic acid (111 mg, 0.67 mmol) in DMF (1 mL) were added DIPEA (0.26 mL) and HATU (391.9 mg, 1.03 mmol) at rt and the reaction was stirred for 16 h at rt. After completion of the reaction, reaction mass was purified by preparative HPLC to yield N-(3-chloro-4-(oxazol-5-yl)phenyl)-2-(3-methoxyphenyl)acetamide (Compound (1)) (46 mg, 26%). UPLC Rt: 1.50 min; MS: 343.1 (M+H).

### Synthesis of Compound (2)

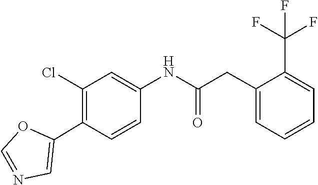 [SMILES: O=C(Cc1ccccc1C(F)(F)F)Nc1ccc(-c2cnco2)c(Cl)c1]

### N-(3-chloro-4-(oxazol-5-yl)phenyl)-2-(2-(trifluoromethyl)phenyl)acetamide

To a stirred solution of 3-chloro-4-(oxazole-5-yl)aniline (Intermediate 2) (100 mg, 0.51 mmol) and 2-(2-(trifluoromethyl)phenyl)acetic acid (137 mg, 0.67 mmol) in DMF (1 mL) were added DIPEA (0.26 mL) and HATU (391.9 mg, 1.03 mmol) at rt and the reaction was stirred for 16 h at rt. After completion of the reaction, reaction mass was purified by preparative HPLC to yield N-(3-chloro-4-(oxazol-5-yl)phenyl)-2-(2-(trifluoromethyl)phenyl)acetamide (Compound (2)) (68 mg, 35%). UPLC Rt: 1.74 min; MS: 381.1 (M+H).

### Synthesis of Compound (3)

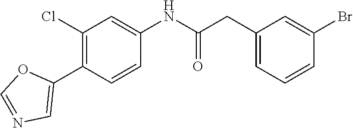 [SMILES: O=C(Cc1cccc(Br)c1)Nc1ccc(-c2cnco2)c(Cl)c1]

### 2-(3-bromophenyl)-N-(3-chloro-4-(oxazol-5-yl)phenyl)acetamide

To a stirred solution of 3-chloro-4-(oxazole-5-yl)aniline (Intermediate 2) (100 mg, 0.51 mmol) and (2-Chloro-5-fluoro-phenyl)-acetic acid (144 mg, 0.67 mmol) in DMF (1 mL) were added DIPEA (0.26 mL) and HATU (391.9 mg, 1.03 mmol) at rt and the reaction was stirred for 16 h at rt. After completion of the reaction, reaction mass was purified by preparative HPLC to yield 2-(3-bromophenyl)-N-(3-chloro-4-(oxazol-5-yl)phenyl)acetamide (Compound (3)) (62 mg, 31%). UPLC Rt: 1.77 min; MS: 393.1 (M+H).

### Synthesis of Compound (4)

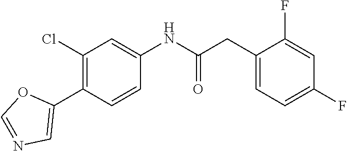 [SMILES: O=C(Cc1ccc(F)cc1F)Nc1ccc(-c2cnco2)c(Cl)c1]

### N-(3-chloro-4-(oxazol-5-yl)phenyl)-2-(2,4-difluorophenyl)acetamide

To a stirred solution of 3-chloro-4-(oxazole-5-yl)aniline (Intermediate 2) (100 mg, 0.51 mmol) and (2-Chloro-5-fluoro-phenyl)-acetic acid (115 mg, 0.67 mmol) in DMF (1 mL) were added DIPEA (0.26 mL) and HATU (391.9 mg, 1.03 mmol) at rt and the reaction was stirred for 16 h at rt. After completion of the reaction, reaction mass was purified by preparative HPLC to yield N-(3-chloro-4-(oxazol-5-yl)phenyl)-2-(2,4-difluorophenyl) acetamide (Compound (4)) (57 mg, 32%). UPLC Rt: 1.59 min; MS: 349.1 (M+H).

### Synthesis of Compound (5)

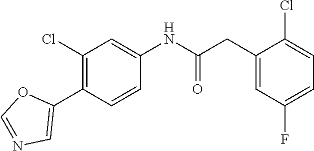 [SMILES: O=C(Cc1cc(F)ccc1Cl)Nc1ccc(-c2cnco2)c(Cl)c1]

### N-(3-chloro-4-(oxazol-5-yl)phenyl)-2-(2-chloro-5-fluorophenyl)acetamide

To a stirred solution of 3-chloro-4-(oxazole-5-yl)aniline (Intermediate 2) (100 mg, 0.51 mmol) and (2-Chloro-5-fluoro-phenyl)-acetic acid (126 mg, 0.67 mmol) in DMF (1 mL) were added DIPEA (0.26 mL) and HATU (391.9 mg, 1.03 mmol) at rt and the reaction was stirred for 16 h at rt. After completion of the reaction, reaction mass was purified by preparative HPLC to yield N-(3-chloro-4-(oxazol-5-yl)phenyl)-2-(2-chloro-5-fluorophenyl)acetamide (Compound (5)) (34 mg, 18%). UPLC Rt: 1.64 min; MS: 365 (M+H).

### Synthesis of Compound (6)

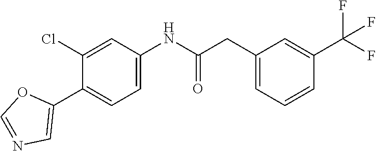 [SMILES: O=C(Cc1cccc(C(F)(F)F)c1)Nc1ccc(-c2cnco2)c(Cl)c1]

### N-(3-chloro-4-(oxazol-5-yl)phenyl)-2-(3-(trifluoromethyl)phenyl)acetamide

To a stirred solution of 3-chloro-4-(oxazole-5-yl)aniline (Intermediate 2) (100 mg, 0.51 mmol) and (3-Trifluoromethyl-phenyl)-acetic acid (136 mg, 0.67 mmol) in DMF (1 mL) were added DIPEA (0.26 mL) and HATU (392 mg, 1.03 mmol) at rt and the reaction was stirred for 16 h at rt. After completion of the reaction, reaction mass was purified by preparative HPLC to yield N-(3-chloro-4-(oxazol-5-yl)phenyl)-2-(3-(trifluoromethyl)phenyl)acetamide (Compound (6)) (26 mg, 13%). UPLC Rt: 1.76 min; MS: 381 (M+H).

### Synthesis of Compound (7)

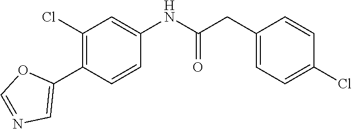 [SMILES: O=C(Cc1ccc(Cl)cc1)Nc1ccc(-c2cnco2)c(Cl)c1]

### N-(3-chloro-4-(oxazol-5-yl)phenyl)-2-(4-chlorophenyl)acetamide

To a stirred solution of 3-chloro-4-(oxazole-5-yl)aniline (Intermediate 2) (100 mg, 0.51 mmol) and (4-chloro-phenyl)-acetic acid (114 mg, 0.67 mmol) in DMF (1 mL) were added DIPEA (0.26 mL) and HATU (391.9 mg, 1.03 mmol) at rt and the reaction was stirred for 16 h at rt. After completion of the reaction, reaction mass was purified by preparative HPLC to yield N-(3-chloro-4-(oxazol-5-yl)phenyl)-2-(4-chlorophenyl)acetamide (Compound (7)) (21 mg, 11%). UPLC Rt: 1.68 min; MS: 347.2 (M+H).

### Synthesis of Compound (8)

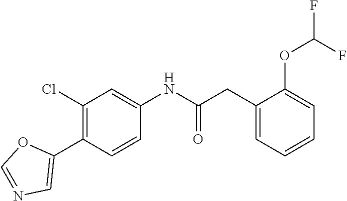 [SMILES: O=C(Cc1ccccc1OC(F)F)Nc1ccc(-c2cnco2)c(Cl)c1]

### N-(3-chloro-4-(oxazol-5-yl)phenyl)-2-(2-(difluoromethoxy)phenyl)acetamide

To a stirred solution of 3-chloro-4-(oxazole-5-yl)aniline (Intermediate 2) (150 mg, 0.77 mmol) and 2-(difluoromethoxy) phenyl acetic acid (203 mg, 1 mmol) in DMF (1.5 mL) were added DIPEA (0.39 mL) and HATU (588 mg, 1.55 mmol) at rt and the reaction was stirred for 16 h at rt. After completion of the reaction, reaction mass was purified by preparative HPLC to yield N-(3-chloro-4-(oxazol-5-yl)phenyl)-2-(2-(difluoromethoxy)phenyl)acetamide (Compound (8)) (56 mg, 19%). UPLC Rt: 1.59 min; MS: 379.2 (M+H).

### Synthesis of Compound (9)

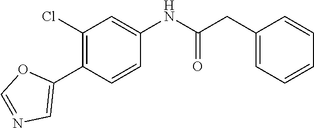 [SMILES: O=C(Cc1ccccc1)Nc1ccc(-c2cnco2)c(Cl)c1]

### N-(3-chloro-4-(oxazol-5-yl)phenyl)-2-phenylacetamide

To a stirred solution of 3-chloro-4-(oxazole-5-yl)aniline (Intermediate 2) (150 mg, 0.77 mmol) and phenyl acetic acid (136.85 mg, 1.00 mmol) in DMF (1.5 mL) were added DIPEA (0.39 mL) and HATU (588 mg, 1.55 mmol) at rt and the reaction was stirred for 16 h at rt. After completion of the reaction, reaction mass was purified by preparative HPLC to yield N-(3-chloro-4-(oxazol-5-yl)phenyl)-2-phenylacetamide (Compound (9)) (69 mg, 28%). UPLC Rt: 1.46 min; MS: 313.2 (M+H).

### Synthesis of Compound (10)

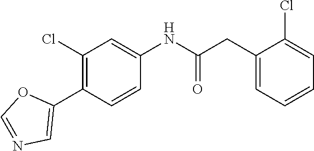 [SMILES: O=C(Cc1ccccc1Cl)Nc1ccc(-c2cnco2)c(Cl)c1]

### N-(3-chloro-4-(oxazol-5-yl)phenyl)-2-(2-chlorophenyl)acetamide

To a stirred solution of 3-chloro-4-(oxazole-5-yl)aniline (Intermediate 2) (150 mg, 0.77 mmol) and 2-chlorophenylacetic acid (171 mg, 1.00 mmol) in DMF (1.5 mL) were added DIPEA (0.39 mL) and HATU (588 mg, 1.55 mmol) at rt and the reaction was stirred for 16 h at rt. After completion of the reaction, reaction mass was purified by preparative HPLC to yield N-(3-chloro-4-(oxazol-5-yl)phenyl)-2-(2-chlorophenyl)acetamide (Compound (10)) (73 mg, 27%). UPLC Rt: 1.59 min; MS: 347.1 (M+H).

### Synthesis of Compound (11)

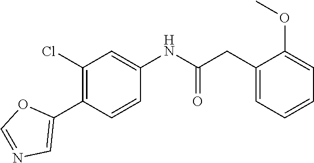 [SMILES: COc1ccccc1CC(=O)Nc1ccc(-c2cnco2)c(Cl)c1]

### N-(3-chloro-4-(oxazol-5-yl)phenyl)-2-(2-methoxyphenyl)acetamide

To a stirred solution of 3-chloro-4-(oxazole-5-yl)aniline (Intermediate 2) (150 mg, 0.77 mmol) and 2-methoxy-phenylacetic acid (167 mg, 1.00 mmol) in DMF (1.5 mL) were added DIPEA (0.39 mL) and HATU (588 mg, 1.55 mmol) at rt and the reaction was stirred for 16 h at rt. After completion of the reaction, reaction mass was purified by preparative HPLC to yield N-(3-chloro-4-(oxazol-5-yl)phenyl)-2-(2-methoxyphenyl)acetamide (Compound (11)) (84 mg, 30%). UPLC Rt: 1.48 min; MS: 343.2 (M+H).

### Synthesis of Compound (12)

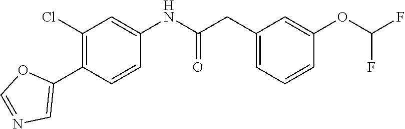 [SMILES: O=C(Cc1cccc(OC(F)F)c1)Nc1ccc(-c2cnco2)c(Cl)c1]

### N-(3-chloro-4-(oxazol-5-yl)phenyl)-2-(3-(difluoromethoxy)phenyl)acetamide

To a stirred solution of 3-chloro-4-(oxazole-5-yl)aniline (Intermediate 2) (200 mg, 1.03 mmol) and 3-(difluoromethoxy)phenyl acetic acid (270.93 mg, 1.34 mmol) in DMF (2 mL) were added DIPEA (0.52 mL) and HATU (784 mg, 2.06 mmol) at rt and the reaction was stirred for 16 h at rt. After completion of the reaction, reaction mass was purified by preparative HPLC to yield N-(3-chloro-4-(oxazol-5-yl)phenyl)-2-(3-(difluoromethoxy)phenyl)acetamide (Compound (12)) (34 mg, 18%). UPLC Rt: 1.66 min; MS: 379.2 (M+H).

### Synthesis of Compound (13)

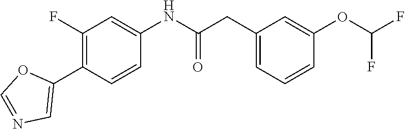 [SMILES: O=C(Cc1cccc(OC(F)F)c1)Nc1ccc(-c2cnco2)c(F)c1]

### 2-(3-(difluoromethoxy)phenyl)-N-(3-fluoro-4-(oxazol-5-yl)phenyl)acetamide

To a stirred solution of 3-fluoro-4-(oxazole-5-yl)aniline (Intermediate 4) (200 mg, 1.12 mmol) and 2-(3-(difluoromethoxy)phenyl)acetic acid (295.3 mg, 1.46 mmol) in DMF (2 mL) were added DIPEA (0.58 mL) and HATU (854.4 mg, 2.24 mmol) at rt and the reaction was stirred for 16 h at rt. After completion of the reaction, reaction mass was purified by preparative HPLC to yield 2-(3-(difluoromethoxy)phenyl)-N-(3-fluoro-4-(oxazol-5-yl)phenyl)acetamide (Compound (13)) (104 mg, 25%). UPLC Rt: 1.55 min; MS: 363.2 (M+H).

### Synthesis of Compound (14)

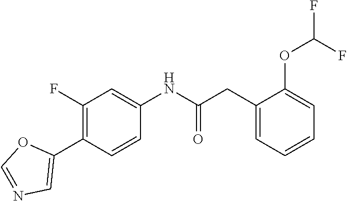 [SMILES: O=C(Cc1ccccc1OC(F)F)Nc1ccc(-c2cnco2)c(F)c1]

### 2-(2-(difluoromethoxy)phenyl)-N-(3-fluoro-4-(oxazol-5-yl)phenyl) acetamide

To a stirred solution of 3-fluoro-4-(oxazole-5-yl)aniline (Intermediate 4) (200 mg, 1.12 mmol) and 2-(difluoromethoxy) phenyl acetic acid (295.3 mg, 1.46 mmol) in DMF (2 mL) were added DIPEA (0.58 mL) and HATU (854.4 mg, 2.24 mmol) at rt and the reaction was stirred for 16 h at rt. After completion of the reaction, reaction mass was purified by preparative HPLC to yield 2-(2-(difluoromethoxy)phenyl)-N-(3-fluoro-4-(oxazol-5-yl)phenyl)acetamide (Compound (14)) (147 mg, 36%). UPLC Rt: 1.50 min; MS: 363.2 (M+H).

### Synthesis of Compound (15)

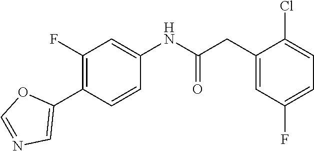 [SMILES: O=C(Cc1cc(F)ccc1Cl)Nc1ccc(-c2cnco2)c(F)c1]

### 2-(2-chloro-5-fluorophenyl)-N-(3-fluoro-4-(oxazol-5-yl)phenyl) acetamide

To a stirred solution of 3-fluoro-4-(oxazole-5-yl)aniline (Intermediate 4) (200 mg, 1.12 mmol) and (2-Chloro-5-fluoro-phenyl)-acetic acid (275.5 mg, 1.46 mmol) in DMF (2 mL) were added DIPEA (0.58 mL) and HATU (854.4 mg, 2.24 mmol) at rt and the reaction was stirred for 16 h at rt. After completion of the reaction, reaction mass was purified by preparative HPLC to yield 2-(2-chloro-5-fluorophenyl)-N-(3-fluoro-4-(oxazol-5-yl)phenyl)acetamide (Compound (15)) (137 mg, 34%). UPLC Rt: 1.57 min; MS: 349.2 (M+H).

### Synthesis of Compound (16)

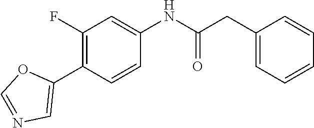 [SMILES: O=C(Cc1ccccc1)Nc1ccc(-c2cnco2)c(F)c1]

### N-(3-fluoro-4-(oxazol-5-yl)phenyl)-2-phenylacetamide

To a stirred solution of 3-fluoro-4-(oxazole-5-yl)aniline (Intermediate 4) (100 mg, 0.56 mmol) and 2-phenylacetic acid (99.4 mg, 0.73 mmol) in DMF (1 mL) were added DIPEA (0.29 mL) and HATU (427 mg, 1.12 mmol) at rt and the reaction was stirred for 16 h at rt. After completion of the reaction, reaction mass was purified by preparative HPLC to yield N-(3-fluoro-4-(oxazol-5-yl)phenyl)-2-phenylacetamide (Compound (16)) (43 mg, 25%). UPLC Rt: 1.36 min; MS: 297.2 (M+H).

### Synthesis of Compound (17)

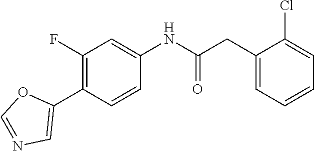 [SMILES: O=C(Cc1ccccc1Cl)Nc1ccc(-c2cnco2)c(F)c1]

### 2-(2-chlorophenyl)-N-(3-fluoro-4-(oxazol-5-yl)phenyl)acetamide

To a stirred solution of 3-fluoro-4-(oxazole-5-yl)aniline (Intermediate 4) (150 mg, 0.84 mmol) and 2-(2-chlorophenyl)acetic acid (186.9 mg, 1.09 mmol) in DMF (1.5 mL) were added DIPEA (0.44 mL) and HATU (641 mg, 1.68 mmol) at rt and the reaction was stirred for 16 h at rt. After completion of the reaction, reaction mass was purified by preparative HPLC to yield 2-(2-chlorophenyl)-N-(3-fluoro-4-(oxazol-5-yl)phenyl)acetamide (Compound (17)) (123 mg, 44%). UPLC Rt: 1.50 min; MS: 331.2 (M+H).

### Synthesis of Compound (18)

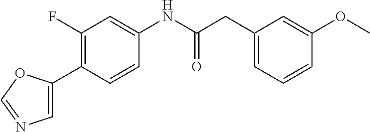 [SMILES: COc1cccc(CC(=O)Nc2ccc(-c3cnco3)c(F)c2)c1]

### N-(3-fluoro-4-(oxazol-5-yl)phenyl)-2-(3-methoxyphenyl)acetamide

To a stirred solution of 3-fluoro-4-(oxazole-5-yl)aniline (Intermediate 4) (150 mg, 0.84 mmol) and 3-methoxy-phenylacetic acid (182.1 mg, 1.09 mmol) in DMF (1.5 mL) were added DIPEA (0.44 mL) and HATU (641 mg, 1.68 mmol) at rt and the reaction was stirred for 16 h at rt. After completion of the reaction, reaction mass was purified by preparative HPLC to yield N-(3-fluoro-4-(oxazol-5-yl)phenyl)-2-(3-methoxyphenyl)acetamide (Compound (18)) (87 mg, 31%). UPLC Rt: 1.37 min; MS: 327.2 (M+H).

### Synthesis of Compound (19)

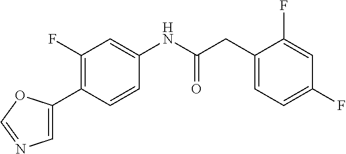 [SMILES: O=C(Cc1ccc(F)cc1F)Nc1ccc(-c2cnco2)c(F)c1]

### 2-(2,4-difluorophenyl)-N-(3-fluoro-4-(oxazol-5-yl)phenyl) acetamide

To a stirred solution of 3-fluoro-4-(oxazole-5-yl)aniline (Intermediate 4) (150 mg, 0.84 mmol) and 2,4-difluorophenyl acetic acid (188.56 mg, 1.09 mmol) in DMF (1.5 mL) were added DIPEA (0.44 mL) and HATU (641 mg, 1.68 mmol) at rt and the reaction was stirred for 16 h at rt. After completion of the reaction, reaction mass was purified by preparative HPLC to yield to get 2-(2,4-difluorophenyl)-N-(3-fluoro-4-(oxazol-5-yl)phenyl)acetamide (Compound (19)) (125 mg, 44%). UPLC Rt: 1.52 min; MS: 333.2 (M+H).

### Synthesis of Compound (20)

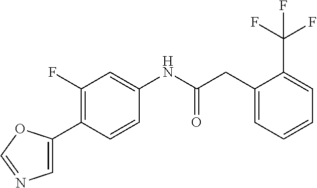 [SMILES: O=C(Cc1ccccc1C(F)(F)F)Nc1ccc(-c2cnco2)c(F)c1]

### N-(3-fluoro-4-(oxazol-5-yl)phenyl)-2-(2-(trifluoromethyl)phenyl)acetamide

To a stirred solution of 3-fluoro-4-(oxazole-5-yl)aniline (Intermediate 4) (150 mg, 0.84 mmol) and 2-(trifluoromethyl)phenyl acetic acid (223.6 mg, 1.09 mmol) in DMF (1.5 mL) were added DIPEA (0.44 mL) and HATU (641 mg, 1.68 mmol) at rt and the reaction was stirred for 16 h at rt. After completion of the reaction, reaction mass was purified by preparative HPLC to yield to get N-(3-fluoro-4-(oxazol-5-yl)phenyl)-2-(2-(trifluoromethyl)phenyl)acetamide (Compound (20)) (125 mg, 40%). UPLC Rt: 1.69 min; MS: 365 (M+H).

### Synthesis of compounds comprising a pyridine-4-yl residue

### Intermediate 5

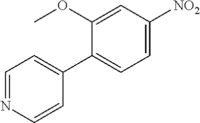 [SMILES: COc1cc([N+](=O)[O-])ccc1-c1ccncc1]

### 4-(2-methoxy-4-nitrophenyl)pyridine

To a stirred solution of 1-bromo-2-methoxy-4-nitrobenzene (5 g, 21.55 mmol) in 1.4 dioxane (50 ml) and water (10 ml) were added (pyridin-4-yl)boronic acid (3.97 g, 32.32 mmol) and K₂CO₃ (8.92 g, 64.65 mmol). After degassing with nitrogen for 10 min Pd(Ph₃P)₄ (0.498 g, 0.431 mmol) was added and the flask was degassed again with nitrogen to then let the reaction mixture be stirred at 85-90° C. for 12 h. After completion of the reaction the reaction mixture was diluted with ethyl acetate (100 ml) followed by washing the ethyl acetate layer with water (2×50 ml) and brine (2×50 ml) successively. The organic layer was dried with Na2SO4 and concentrated to dryness and the crude mass was purified by flash column chromatography, eluted with 15% E.A-Hexane, to afford 4-(2-methoxy-4-nitrophenyl)pyridine (Intermediate 5) (2.5 g, 50.4%) as white solid. LCMS: 230 (M+H).

### Intermediate 6

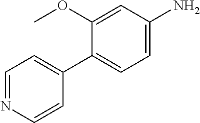

### 3-methoxy-4-(pyridin-4-yl)aniline

A flask containing 4-(2-methoxy-4-nitrophenyl) pyridine (Intermediate 5) (2.5 g, 10.8 mmol) was flushed with N₂ and 10% pd/c (2.3 g, 21.7 mmol) was added. Ethyl acetate (50 mL) was added to the mixture, the N₂ supply was replaced with H2 and the black suspension was stirred under H2 for 5 h after which the reaction was completed. The suspension was filtered through celite, washed with ethyl acetate and concentrated under vacuum to yield 3-methoxy-4-(pyridin-4-yl)aniline (Intermediate 6) (1.42 g, 65.2%) as yellow solid. LCMS: 200 (M+H).

### Synthesis of Compound (21)

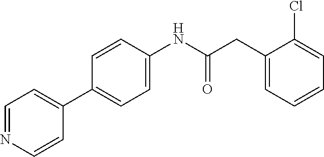 [SMILES: O=C(Cc1ccccc1Cl)Nc1ccc(-c2ccncc2)cc1]

### 2-(2-chlorophenyl)-N-(4-(pyridin-4-yl)phenyl)acetamide

To a stirred solution of 4-(pyridin-4-yl)aniline (75 mg, 0.441 mmol) and 2-(2-chlorophenyl)acetic acid (112.5 mg, 0.66 mmol) in DMF (3 mL) were added DIPEA (0.169 mL) and HATU (252.7 mg, 0.66 mmol) at rt and the reaction was stirred for 12 h at rt. After completion of the reaction, the reaction mixture was purified by preparative HPLC to yield 2-(2-chlorophenyl)-N-(4-(pyridin-4-yl)phenyl)acetamide (Compound (21)) (51.3 mg, 36%). UPLC Rt: 0.92 min; MS: 323.2 (M+H).

### Synthesis of Compound (22)

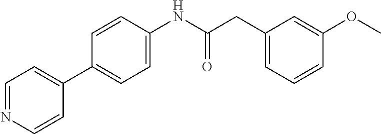 [SMILES: COc1cccc(CC(=O)Nc2ccc(-c3ccncc3)cc2)c1]

### 2-(3-methoxyphenyl)-N-(4-(pyridin-4-yl)phenyl)acetamide

To a stirred solution of 4-(pyridin-4-yl)aniline (75 mg, 0.441 mmol) and 2-(3-methoxyphenyl)acetic acid (110 mg, 0.66 mmol) in DMF (3 mL) were added DIPEA (0.169 mL) and HATU (252.7 mg, 0.66 mmol) at rt and the reaction was stirred for 12 h at rt. After completion of the reaction, the reaction mixture was purified by preparative HPLC to yield 2-(3-methoxyphenyl)-N-(4-(pyridin-4-yl)phenyl)acetamide (Compound (22)) (56 mg, 40%). UPLC Rt: 0.86 min; MS: 319.2 (M+H).

### Synthesis of Compound (23)

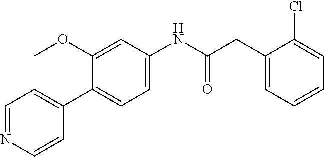 [SMILES: COc1cc(NC(=O)Cc2ccccc2Cl)ccc1-c1ccncc1]

### 2-(2-chlorophenyl)-N-(3-methoxy-4-(pyridin-4-yl)phenyl) acetamide

To a stirred solution of 3-methoxy-4-(pyridin-4-yl)aniline (Intermediate 6) (75 mg, 0.375 mmol) and 2-(2-chlorophenyl)acetic acid (96 mg, 0.563 mmol) in DMF (2 mL) were added DIPEA (0.144 mL) and HATU (214.8 mg, 0.563 mmol) at rt and the reaction was stirred for 12 h at rt. After completion of the reaction, the reaction mixture was purified by preparative HPLC to yield 2-(2-chlorophenyl)-N-(3-methoxy-4-(pyridin-4-yl)phenyl)acetamide (Compound (23)) (20 mg, 15%). UPLC Rt: 0.96 min; MS: 353.25 (M+H).

### Synthesis of Compound (24)

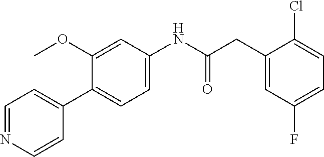 [SMILES: COc1cc(NC(=O)Cc2cc(F)ccc2Cl)ccc1-c1ccncc1]

### 2-(2-chloro-5-fluorophenyl)-N-(3-methoxy-4-(pyridin-4-yl)phenyl) acetamide

To a stirred solution of 3-methoxy-4-(pyridin-4-yl)aniline (Intermediate 6) (75 mg, 0.375 mmol) and 2-(2-chloro-5-fluorophenyl)acetic acid (105.7 mg, 0.563 mmol) in DMF (2 mL) were added DIPEA (0.144 mL) and HATU (214.8 mg, 0.563 mmol) at rt and the reaction was stirred for 12 h at rt. After completion of the reaction, the reaction mixture was purified by preparative HPLC to yield 2-(2-chloro-5-fluorophenyl)-N-(3-methoxy-4-(pyridin-4-yl)phenyl)acetamide (Compound (24)) (69 mg, 50%). UPLC Rt: 1.05 min; MS: 371.2 (M+H).

### Synthesis of Compound (25)

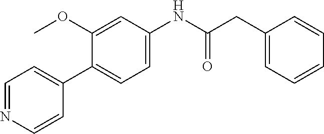 [SMILES: COc1cc(NC(=O)Cc2ccccc2)ccc1-c1ccncc1]

### N-(3-methoxy-4-(pyridin-4-yl)phenyl)-2-phenylacetamide

To a stirred solution of 3-methoxy-4-(pyridin-4-yl)aniline (Intermediate 6) (75 mg, 0.375 mmol) and 2-phenylacetic acid (73.1 mg, 0.563 mmol) in DMF (2 mL) were added DIPEA (0.144 mL) and HATU (214.8 mg, 0.563 mmol) at rt and the reaction was stirred for 12 h at rt. After completion of the reaction, the reaction mixture was purified by preparative HPLC to yield N-(3-methoxy-4-(pyridin-4-yl)phenyl)-2-phenylacetamide (Compound (25)) (46 mg, 39%). UPLC Rt: 0.88 min; MS: 319.2 (M+H).

### Synthesis of Compound (26)

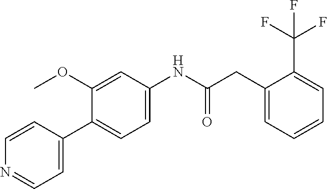 [SMILES: COc1cc(NC(=O)Cc2ccccc2C(F)(F)F)ccc1-c1ccncc1]

### N-(3-methoxy-4-(pyridin-4-yl)phenyl)-2-(2-(trifluoromethyl)phenyl)acetamide

To a stirred solution of 3-methoxy-4-(pyridin-4-yl)aniline (Intermediate 6) (75 mg, 0.375 mmol) and 2-(2-(trifluoromethyl)phenyl)acetic acid (114.9 mg, 0.563 mmol) in DMF (2 mL) were added DIPEA (0.144 mL) and HATU (214.8 mg, 0.563 mmol) at rt and the reaction was stirred for 12 h at rt. After completion of the reaction, the reaction mixture was purified by preparative HPLC to N-(3-methoxy-4-(pyridin-4-yl)phenyl)-2-(2-(trifluoromethyl)phenyl)acetamide (Compound (26)) (118 mg, 81%). UPLC Rt: 1.13 min; MS: 387.3 (M+H).

### Synthesis of Compound (27)

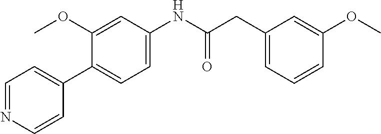 [SMILES: COc1cccc(CC(=O)Nc2ccc(-c3ccncc3)c(OC)c2)c1]

### N-(3-methoxy-4-(pyridin-4-yl)phenyl)-2-(3-methoxyphenyl)acetamide

To a stirred solution of 3-methoxy-4-(pyridin-4-yl)aniline (Intermediate 6) (75 mg, 0.375 mmol) and 2-(3-methoxyphenyl)acetic acid (93.3 mg, 0.563 mmol) in DMF (2 mL) were added DIPEA (0.144 mL) and HATU (214.8 mg, 0.563 mmol) at rt and the reaction was stirred for 12 h at rt. After completion of the reaction, the reaction mixture was purified by preparative HPLC to yield N-(3-methoxy-4-(pyridin-4-yl)phenyl)-2-(3-methoxyphenyl)acetamide (Compound (27)) (68 mg, 52%). UPLC Rt: 0.91 min; MS: 349.3 (M+H).

### Synthesis of Compound (28)

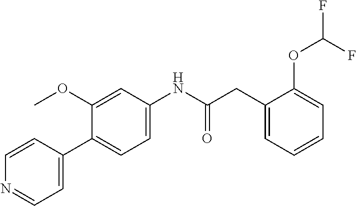 [SMILES: COc1cc(NC(=O)Cc2ccccc2OC(F)F)ccc1-c1ccncc1]

### 2-(2-(difluoromethoxy)phenyl)-N-(3-methoxy-4-(pyridin-4-yl)phenyl) acetamide

To a stirred solution of 3-methoxy-4-(pyridin-4-yl)aniline (Intermediate 6) (75 mg, 0.375 mmol) and 2-(2-(difluoromethoxy)phenyl)acetic acid (113.6 mg, 0.563 mmol) in DMF (2 mL) were added DIPEA (0.144 mL) and HATU (214.8 mg, 0.563 mmol) at rt and the reaction was stirred for 12 h at rt. After completion of the reaction, the reaction mixture was purified by preparative HPLC to yield 2-(2-(difluoromethoxy)phenyl)-N-(3-methoxy-4-(pyridin-4-yl)phenyl)acetamide (Compound (28)) (75.3 mg, 52%). UPLC Rt: 1.03 min; MS: 385.3 (M+H).

### Preparation of Dissecting Solutions and Enzyme Solutions

Kynurenic Acid (0.2 mg/mL), trypsin (1.33 mg/mL), and hyaluronidase (0.67 mg/mL) were weighed out and dissolved in high magnesium/low calcium artificial cerebral spinal fluid (aCSF) at 37° C. Fibroblast growth factor 2 (FGF2; 10 ng/mL) and heparin (2 μg/mL) were added to 100 mL of serum-free media (SFM). Ovomucoid trypsin inhibitor (1 mg/mL) was dissolved in warm SFM and sterile filtered (22 μm).

### Isolation of Retinal Precursor Cells from the Ciliary Epithelium of the Eye and Primary Sphere Assay

A dissecting microscope, cold light source, and sterile surgical instruments were set up inside of a sterile biological safety cabinet (BSC). Mammalian eyes were enucleated and placed in a petri dish containing cold, sterile aCSF. Under the dissecting microscope, hair, connective tissue, and the dorsal and ventral oblique muscles were cleared from the scleral/corneal border with two sets of forceps. Next, curved or angled micro-dissecting scissors were used to cleave any remaining extraocular muscle tissue, the optic nerve, and cut the eyeball into symmetrical halves; beginning and finishing the cut from the hole left by the optic nerve. Using two sets of forceps to grasp the cornea, the two eye halves were peeled apart. The lens, optic nerve, and vitreous were separated from the eye shells and the eye shells were transferred into a new petri dish (also containing cold, sterile aCSF). To isolate the ciliary epithelium (CE), eye shells were oriented with the cornea on the right and retinal pigmented epithelium (RPE) on the left. A pair of straight forceps were used to pin down the eye shell on the RPE side while a scalpel blade was inserted between the CE and the iris, using pressure to slice the iris/cornea side off from the rest of the shell. Next, the scalpel was run along the border between the CE and the RPE to obtain the CE isolated as a thin strip of tissue. The CE strips were then transferred to a 35 mm dish containing 2 mL of dispase solution (Sigma; T1005) and incubated for 10 minutes at 37° C. Next, the strips were transferred from dispase into a 35 mm dish containing 2 mL of sterile filtered kynurenic acid, trypsin and hyaluronidase solution and incubated at 37° C. for 10 minutes. After incubation, the dish was returned to the dissecting scope, and the CE strips were pinned down with straight, non-serrated forceps, while non-serrated curved forceps were used to scrape the CE off from the underlying sclera. The bare scleral strips were then discarded, such that only the CE cells remained in the enzyme solution. Using a fire-polished, cotton-plugged glass pipette, the cells and enzyme solution were transferred to a 15 mL tube and triturated approximately 45 times to break apart the tissue. The 15 mL tube/cell suspension was centrifuged for 5 minutes at 1500 rpm. The supernatant was gently aspirated from the resulting pellet using a fire-polished, cotton-plugged glass pipette and 2 mL of trypsin inhibitor solution was added to the pellet. Using a small borehole, fire-polished, cotton-plugged glass pipette, the sample was triturated approximately 45 times until it was a single-cell suspension. The 15 mL tube/cell suspension was centrifuged for 5 minutes at 1500 rpm. The supernatant was gently aspirated from the resulting pellet and 1-2 mL of SFM with FGF2 and heparin (plating media) was added. The cells and media were mixed to ensure a uniform cell suspension and a 10 uL sample was taken and cell density was determined. The cells were then seeded and cultured at 10c/μL in culture-treated plates or flasks. After one week, roughly 1 in 500 cells proliferated to form free-floating, clonal spheres greater than 80 μm in diameter.

### Sphere Passaging and High-Throughput Drug Screening

Human-derived spheres were passaged using the kynurenic acid, trypsin, hyaluronidase enzyme solution with the addition of collagenase I (0.5 mg/mL), collagenase II (0.5 mg/mL) and elastase (0.1 mg/mL). Mouse-derived spheres were passaged using hyaluronidase (0.67 mg/mL), collagenase I (0.5 mg/mL), and collagenase II (0.5 mg/mL) dissolved in Accustase solution (Sigma; SCR005). Spheres were collected en masse from culture plates or flasks, transferred into one or more 50 mL tubes and centrifuged for 5 minutes at 1500 rpm. The supernatant was gently aspirated from the pellet and 2-5 mL of enzyme solution was added to the pellet and mixed thoroughly. The 2-5 mL enzyme and sphere suspension was transferred to a 15 mL tube and laid horizontally on an automated rocker at 37° C. for 45 minutes. After incubation, the enzyme solution with spheres was triturated approximately 45 times to mechanically dissociate the spheres. The cell suspension was centrifuged for 5 minutes at 1500 rpm. The supernatant was gently aspirated and 1-2 mL of trypsin inhibitor solution was added to the pellet and triturated approximately 45 times. The cell suspension was centrifuged for 5 minutes at 1500 rpm. The supernatant was gently aspirated from the resulting pellet and 1-2 mL of SFM with FGF2 and heparin (plating media) was added. The cells and media were mixed to ensure a uniform cell suspension and a 10 uL sample was taken and cell density was determined from that sample. The remaining cells were then seeded and cultured at 10c/μL in prepared 96-well or 24-well plates with 0.1% DMSO or a selected concentration of drug in 0.1% DMSO. Cells were grown for one week and then live stained for nuclei (Hoechst 33258; 10 μg/mL). For mouse tissue, an actin-green fluorescent protein (GFP) transgenic mouse strain (FVB.Cg-Tg(CAG-EGFP)B5Nagy/J) was used and cell number comparisons were made based on nuclei and GFP-based quantification. For human tissue, the green fluorescent cell viability dye, calcein AM (ThermoFisher C3100MP; 2 μM) was used and cell number comparisons were made based on nuclei and calcein fluorescence-based quantification.

### Statistical Evaluation of Drug Screening Results

Statistic significance was evaluated on a plate to plate basis employing control wells with no drug treatment and equivalent concentration of DMSO in the medium. The minimal number of control wells was 8 for 96 well plates and 6 for 24 well plates. Average and standard deviations were determined and compound wells with cell numbers outside the three standard deviations range around the control value were classified as hits. Individual compound treatment conditions on each plate were always at least present in duplicates to internally verify the validity of results. Numerical values were then averaged for each compound.

### Results

TABLE 4

| | | Cell |
|---|---|---|
| | | proliferation |
| | | within one |
| Comp. No. | Chemical structure | week [%] |
| 1 | 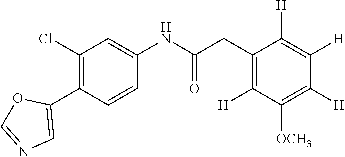 [SMILES: COc1cccc(CC(=O)Nc2ccc(-c3cnco3)c(Cl)c2)c1] | 203 |
| 2 | 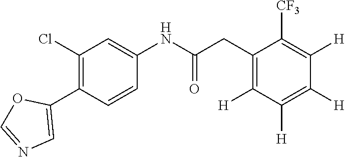 [SMILES: O=C(Cc1ccccc1C(F)(F)F)Nc1ccc(-c2cnco2)c(Cl)c1] | 142 |
| 3 | 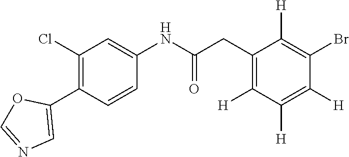 [SMILES: O=C(Cc1cccc(Br)c1)Nc1ccc(-c2cnco2)c(Cl)c1] | 143 |
| 4 | 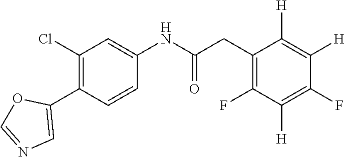 [SMILES: O=C(Cc1ccc(F)cc1F)Nc1ccc(-c2cnco2)c(Cl)c1] | 157 |
| 5 | 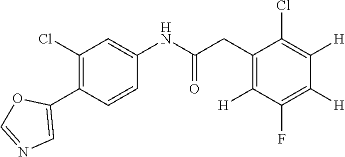 [SMILES: O=C(Cc1cc(F)ccc1Cl)Nc1ccc(-c2cnco2)c(Cl)c1] | 121 |
| 6 | 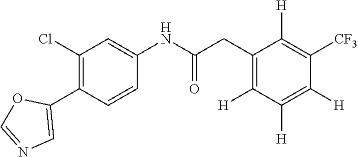 [SMILES: O=C(Cc1cccc(C(F)(F)F)c1)Nc1ccc(-c2cnco2)c(Cl)c1] | 134 |
| 7 | 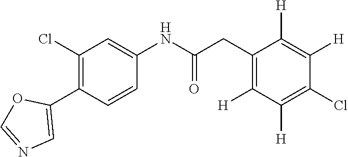 [SMILES: O=C(Cc1ccc(Cl)cc1)Nc1ccc(-c2cnco2)c(Cl)c1] | 133 |
| 8 | 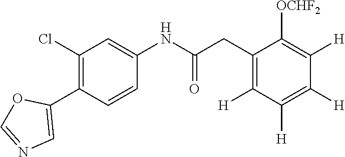 [SMILES: O=C(Cc1ccccc1OC(F)F)Nc1ccc(-c2cnco2)c(Cl)c1] | 135 |
| 9 | 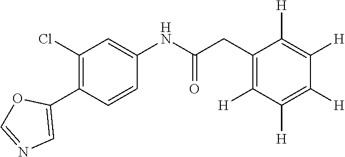 [SMILES: O=C(Cc1ccccc1)Nc1ccc(-c2cnco2)c(Cl)c1] | 149 |
| 10 | 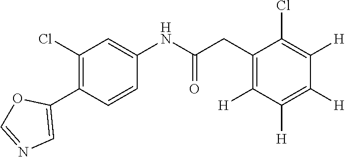 [SMILES: O=C(Cc1ccccc1Cl)Nc1ccc(-c2cnco2)c(Cl)c1] | 158 |
| 11 | 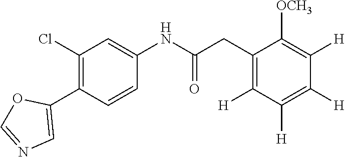 [SMILES: COc1ccccc1CC(=O)Nc1ccc(-c2cnco2)c(Cl)c1] | 128 |
| 12 | 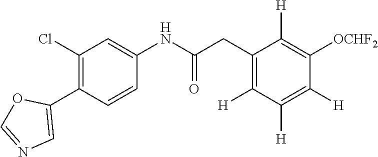 [SMILES: O=C(Cc1cccc(OC(F)F)c1)Nc1ccc(-c2cnco2)c(Cl)c1] | 122 |
| 13 | 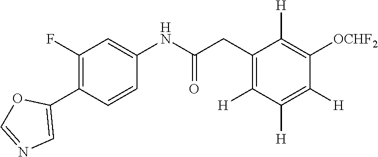 [SMILES: O=C(Cc1cccc(OC(F)F)c1)Nc1ccc(-c2cnco2)c(F)c1] | 120 |
| 14 | 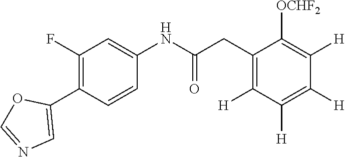 [SMILES: O=C(Cc1ccccc1OC(F)F)Nc1ccc(-c2cnco2)c(F)c1] | 119 |
| 15 | 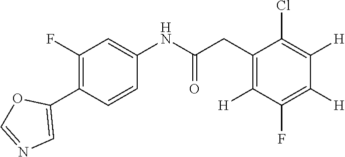 [SMILES: O=C(Cc1cc(F)ccc1Cl)Nc1ccc(-c2cnco2)c(F)c1] | 133 |
| 16 | 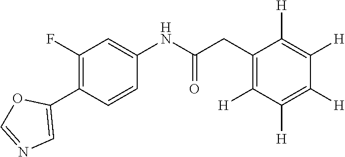 [SMILES: O=C(Cc1ccccc1)Nc1ccc(-c2cnco2)c(F)c1] | 123 |
| 17 | 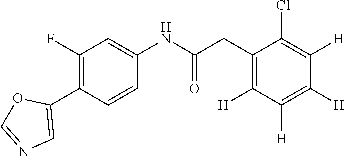 [SMILES: O=C(Cc1ccccc1Cl)Nc1ccc(-c2cnco2)c(F)c1] | 137 |
| 18 | 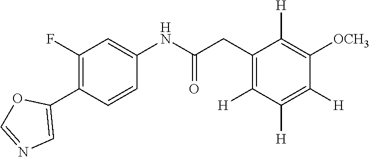 [SMILES: COc1cccc(CC(=O)Nc2ccc(-c3cnco3)c(F)c2)c1] | 127 |
| 19 | 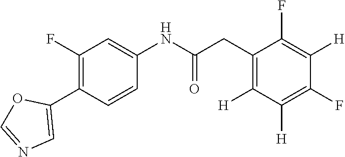 [SMILES: O=C(Cc1ccc(F)cc1F)Nc1ccc(-c2cnco2)c(F)c1] | 140 |
| 20 | 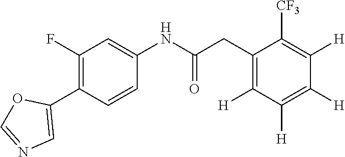 [SMILES: O=C(Cc1ccccc1C(F)(F)F)Nc1ccc(-c2cnco2)c(F)c1] | 118 |
| 21 | 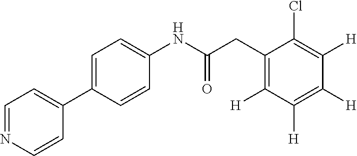 [SMILES: O=C(Cc1ccccc1Cl)Nc1ccc(-c2ccncc2)cc1] | 144 |
| 22 | 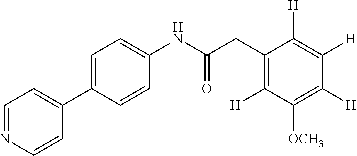 [SMILES: COc1cccc(CC(=O)Nc2ccc(-c3ccncc3)cc2)c1] | 135 |
| 23 | 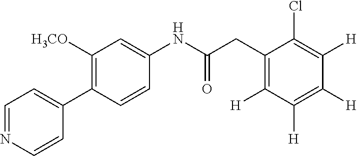 [SMILES: COc1cc(NC(=O)Cc2ccccc2Cl)ccc1-c1ccncc1] | 148 |
| 24 | 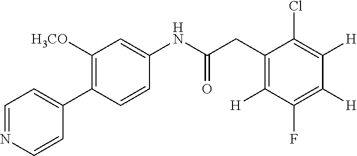 [SMILES: COc1cc(NC(=O)Cc2cc(F)ccc2Cl)ccc1-c1ccncc1] | 134 |
| 25 | 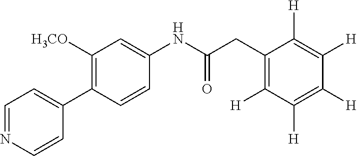 [SMILES: COc1cc(NC(=O)Cc2ccccc2)ccc1-c1ccncc1] | 130 |
| 26 | 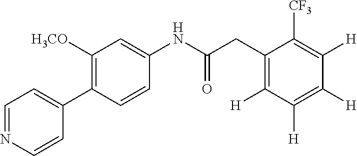 [SMILES: COc1cc(NC(=O)Cc2ccccc2C(F)(F)F)ccc1-c1ccncc1] | 113 |
| 27 | 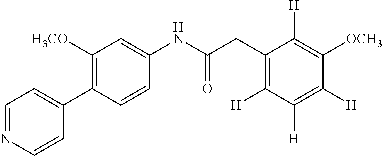 [SMILES: COc1cccc(CC(=O)Nc2ccc(-c3ccncc3)c(OC)c2)c1] | 140 |
| 28 | 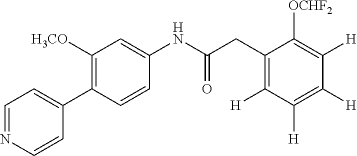 [SMILES: COc1cc(NC(=O)Cc2ccccc2OC(F)F)ccc1-c1ccncc1] | 121 |
| C* | — | 100 |

C* = Control experiment (absence of a compound according to the present invention

1. A compound of the formula (I) 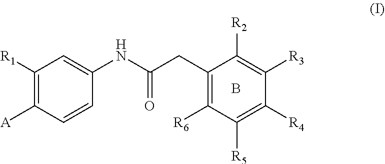
or a pharmaceutically acceptable salt thereof,
wherein:
A is a 5-oxazolyl residue or a pyridine-4-yl residue
R₁ is selected from the group consisting of fluoro and chloro;
R₂, R₃, R₄, R₅ and R₆ of the phenyl ring B are independently from each other selected from the group consisting of hydrogen, a linear or branched alkyl having 1 to 4 carbon atoms, trifluoromethyl, 2,2,2-trifluoroethyl, methylsulfanyl, ethylsulfanyl, methylsulfonyl, ethylsulfonyl, difluoromethoxy, trifluoromethoxy, fluoro, bromo, chloro, methoxy, ethoxy, propoxy, butoxy, hydroxy and amino; and
at least two of R₂, R₃, R₄, R₅ and R₆ are hydrogens, with the proviso that if R₁ is chloro, R₅ is not methoxy.

2. The compound according to claim 1, wherein R₁ is chloro.

3. The compound according to claim 1, wherein A is a 5-oxazolyl residue.

4. The compound according to claim 1, wherein the phenyl ring B is monosubstituted or disubstituted.

5. The compound according to claim 1, wherein the phenyl ring B is monosubstituted.

6. The compound according to claim 5, wherein R₂ is selected from the group consisting of methyl, trifluoromethyl, methylsulfanyl, methylsulfonyl, difluoromethoxy, fluoro, bromo, chloro, methoxy and ethoxy.

7. The compound according to claim 5, wherein R₃ or R₄ is selected from the group consisting of trifluoromethyl, difluoromethoxy, methoxy.

8. The compound according to claim 1, wherein the phenyl ring B is disubstituted.

9. The compound according to claim 8, wherein R₂ is selected from the group consisting of fluoro, bromo, and chloro, and one of R₃, R₄ or R₅ is selected from the group consisting of fluoro, bromo, and chloro.

10. The compound according to claim 9, wherein R₂ is chloro and R₅ is fluoro or wherein both R₂ and R₄ are fluoro.

11. Compound of formula (I) according to claim 1, wherein said compound is selected from the group consisting of

| Comp. | |
|---|---|
| No. | Chemical structure |
| 1 | 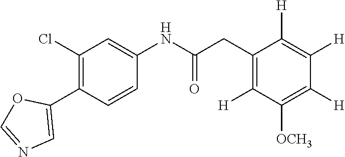 [SMILES: COc1cccc(CC(=O)Nc2ccc(-c3cnco3)c(Cl)c2)c1] |
| 2 | 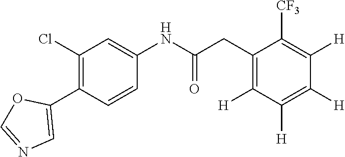 [SMILES: O=C(Cc1ccccc1C(F)(F)F)Nc1ccc(-c2cnco2)c(Cl)c1] |
| 3 | 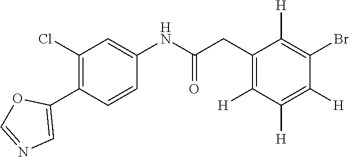 [SMILES: O=C(Cc1cccc(Br)c1)Nc1ccc(-c2cnco2)c(Cl)c1] |
| 4 | 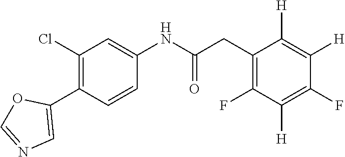 [SMILES: O=C(Cc1ccc(F)cc1F)Nc1ccc(-c2cnco2)c(Cl)c1] |
| 5 | 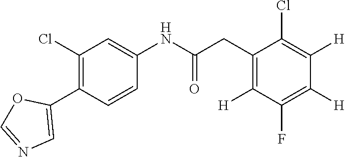 [SMILES: O=C(Cc1cc(F)ccc1Cl)Nc1ccc(-c2cnco2)c(Cl)c1] |
| 6 | 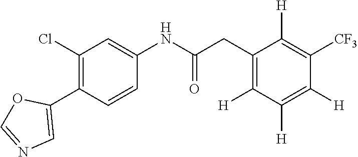 [SMILES: O=C(Cc1cccc(C(F)(F)F)c1)Nc1ccc(-c2cnco2)c(Cl)c1] |
| 7 | 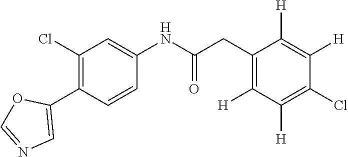 [SMILES: O=C(Cc1ccc(Cl)cc1)Nc1ccc(-c2cnco2)c(Cl)c1] |
| 8 | 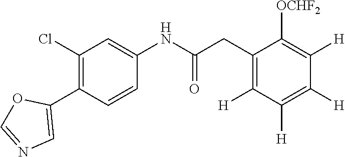 [SMILES: O=C(Cc1ccccc1OC(F)F)Nc1ccc(-c2cnco2)c(Cl)c1] |
| 9 | 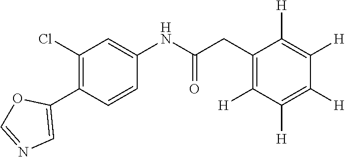 [SMILES: O=C(Cc1ccccc1)Nc1ccc(-c2cnco2)c(Cl)c1] |
| 10 | 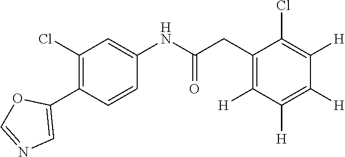 [SMILES: O=C(Cc1ccccc1Cl)Nc1ccc(-c2cnco2)c(Cl)c1] |
| 11 | 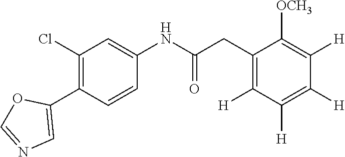 [SMILES: COc1ccccc1CC(=O)Nc1ccc(-c2cnco2)c(Cl)c1] |
| 12 | 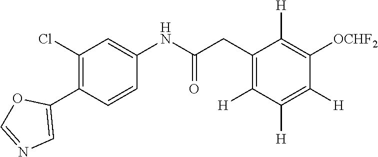 [SMILES: O=C(Cc1cccc(OC(F)F)c1)Nc1ccc(-c2cnco2)c(Cl)c1] |
| 13 | 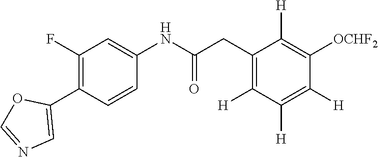 [SMILES: O=C(Cc1cccc(OC(F)F)c1)Nc1ccc(-c2cnco2)c(F)c1] |
| 14 | 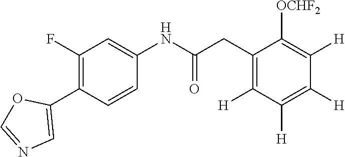 [SMILES: O=C(Cc1ccccc1OC(F)F)Nc1ccc(-c2cnco2)c(F)c1] |
| 15 | 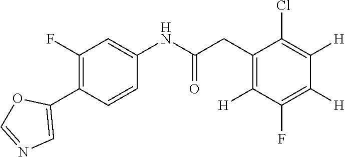 [SMILES: O=C(Cc1cc(F)ccc1Cl)Nc1ccc(-c2cnco2)c(F)c1] |
| 16 | 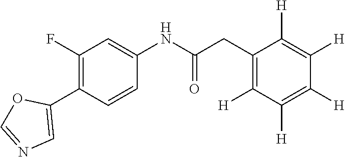 [SMILES: O=C(Cc1ccccc1)Nc1ccc(-c2cnco2)c(F)c1] |
| 17 | 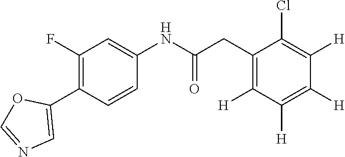 [SMILES: O=C(Cc1ccccc1Cl)Nc1ccc(-c2cnco2)c(F)c1] |
| 18 | 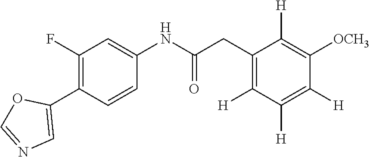 [SMILES: COc1cccc(CC(=O)Nc2ccc(-c3cnco3)c(F)c2)c1] |
| 19 | 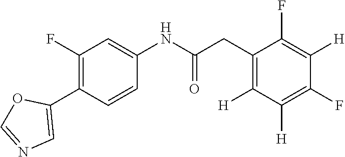 [SMILES: O=C(Cc1ccc(F)cc1F)Nc1ccc(-c2cnco2)c(F)c1] |
| 20 | 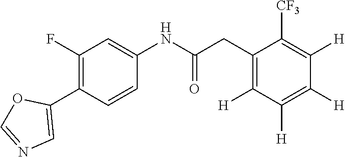 [SMILES: O=C(Cc1ccccc1C(F)(F)F)Nc1ccc(-c2cnco2)c(F)c1] |

12. A pharmaceutical composition comprising a compound of the formula (Ia) for use in the treatment and/or prevention of a primary neuroretinal disease that leads to photoreceptor loss or degradation of the photoreceptor layer of the retina. 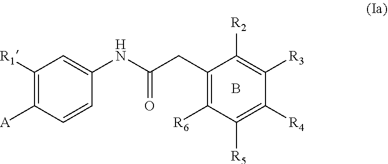
A is a 5-oxazolyl residue or a pyridine-4-yl residue
R₁′ is selected from the group consisting of methoxy, hydrogen, fluoro and chloro;
R₂, R₃, R₄, R₅ and R₆ of the phenyl ring B are independently from each other selected from the group consisting of hydrogen, a linear or branched alkyl having 1 to 4 carbon atoms, trifluoromethyl, 2,2,2-trifluoroethyl, methylsulfanyl, ethylsulfanyl, methylsulfonyl, ethylsulfonyl, difluoromethoxy, trifluoromethoxy, fluoro, bromo, chloro, methoxy, ethoxy, propoxy, butoxy, hydroxy and amino; and
at least two of R₂, R₃, R₄, R₅ and R₆ are hydrogens,
with the proviso that if R₁′ is hydrogen or methoxy, A is a pyridine-4-yl residue,
as a therapeutically active substance.

13. Composition for use according to claim 12, wherein the use is selected from the group consisting of inherited retinal dystrophies, acquired or drug-induced photoreceptor degeneration, infectious eye diseases and inflammatory eye diseases, wherein the pharmaceutical composition, upon administration, treats the retinal disease by inducing proliferation of retinal precursor cells,

14. Composition for use according to claim 12 in the treatment and/or prevention of inherited retinal dystrophies, preferably for use in the treatment of retinitis pigmentosa (RP).

15. Composition for the use according to claim 11 selected from the group consisting of

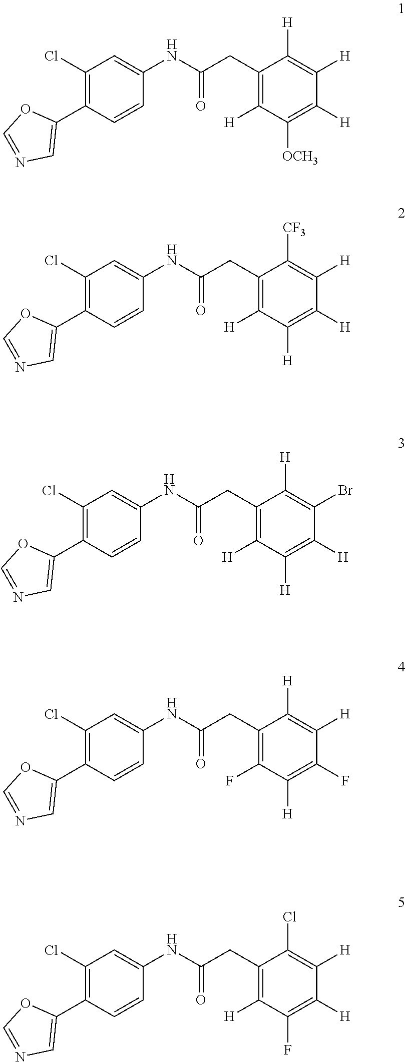 [SMILES: COc1cccc(CC(=O)Nc2ccc(-c3cnco3)c(Cl)c2)c1 ; O=C(Cc1ccccc1C(F)(F)F)Nc1ccc(-c2cnco2)c(Cl)c1 ; O=C(Cc1cccc(Br)c1)Nc1ccc(-c2cnco2)c(Cl)c1 ; O=C(Cc1ccc(F)cc1F)Nc1ccc(-c2cnco2)c(Cl)c1 ; O=C(Cc1cc(F)ccc1Cl)Nc1ccc(-c2cnco2)c(Cl)c1]

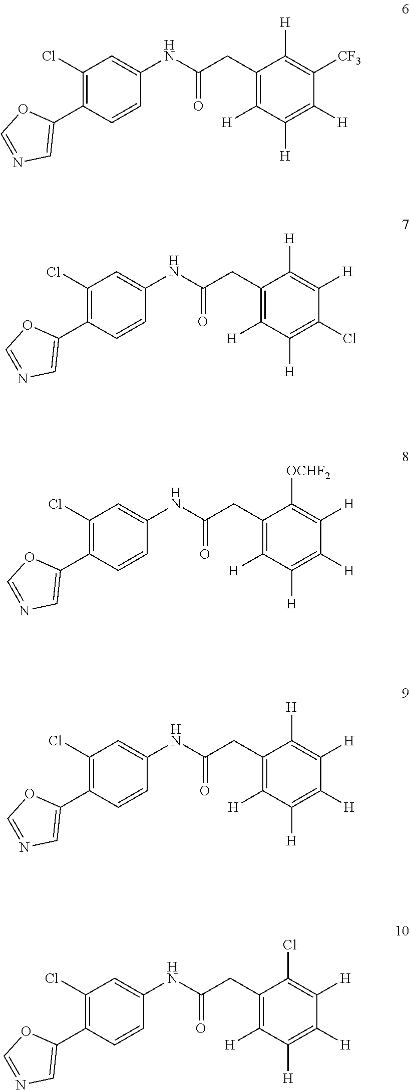 [SMILES: O=C(Cc1cccc(C(F)(F)F)c1)Nc1ccc(-c2cnco2)c(Cl)c1 ; O=C(Cc1ccc(Cl)cc1)Nc1ccc(-c2cnco2)c(Cl)c1 ; O=C(Cc1ccccc1OC(F)F)Nc1ccc(-c2cnco2)c(Cl)c1 ; O=C(Cc1ccccc1)Nc1ccc(-c2cnco2)c(Cl)c1 ; O=C(Cc1ccccc1Cl)Nc1ccc(-c2cnco2)c(Cl)c1]

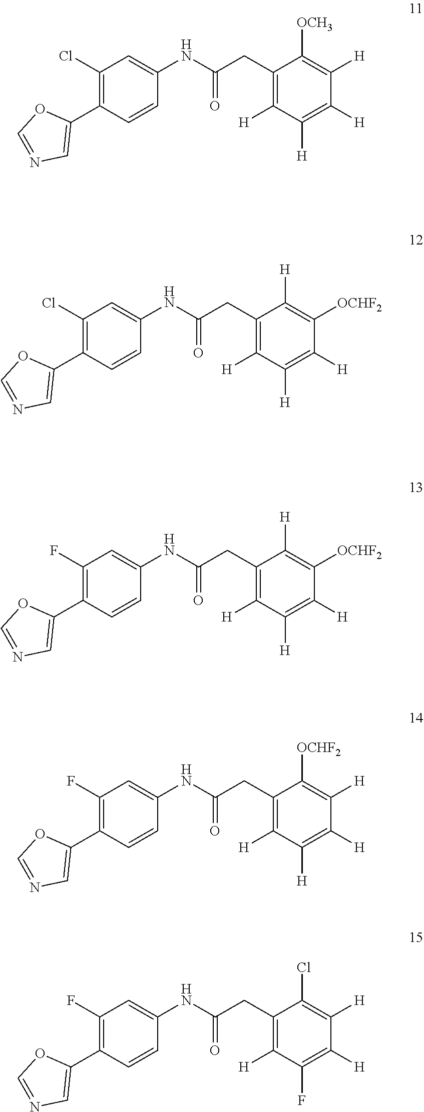 [SMILES: COc1ccccc1CC(=O)Nc1ccc(-c2cnco2)c(Cl)c1 ; O=C(Cc1cccc(OC(F)F)c1)Nc1ccc(-c2cnco2)c(Cl)c1 ; O=C(Cc1cccc(OC(F)F)c1)Nc1ccc(-c2cnco2)c(F)c1 ; O=C(Cc1ccccc1OC(F)F)Nc1ccc(-c2cnco2)c(F)c1 ; O=C(Cc1cc(F)ccc1Cl)Nc1ccc(-c2cnco2)c(F)c1]

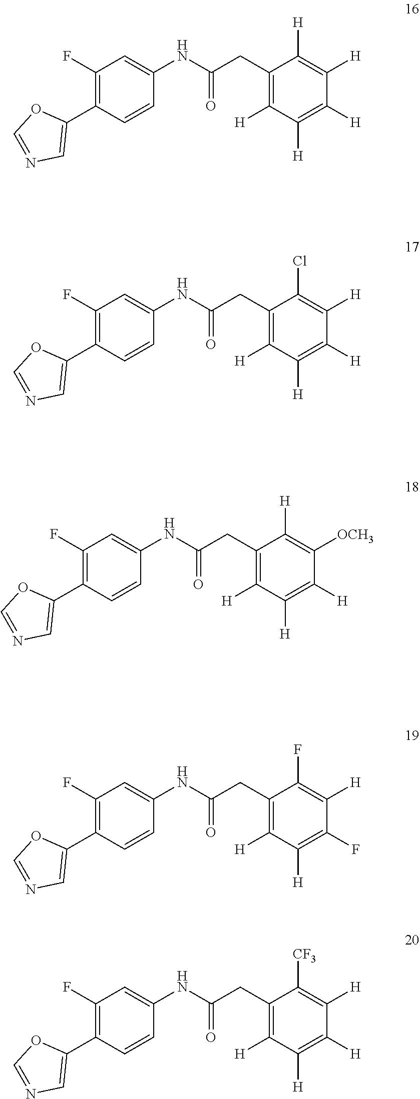 [SMILES: O=C(Cc1ccccc1)Nc1ccc(-c2cnco2)c(F)c1 ; O=C(Cc1ccccc1Cl)Nc1ccc(-c2cnco2)c(F)c1 ; COc1cccc(CC(=O)Nc2ccc(-c3cnco3)c(F)c2)c1 ; O=C(Cc1ccc(F)cc1F)Nc1ccc(-c2cnco2)c(F)c1 ; O=C(Cc1ccccc1C(F)(F)F)Nc1ccc(-c2cnco2)c(F)c1]

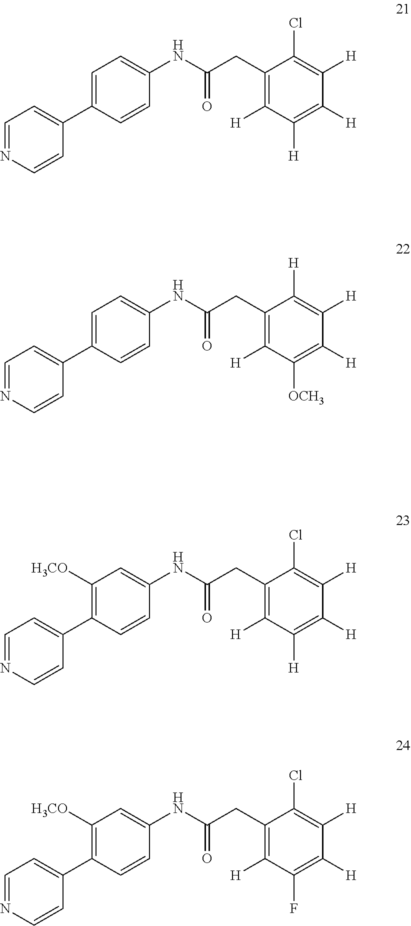 [SMILES: O=C(Cc1ccccc1Cl)Nc1ccc(-c2ccncc2)cc1 ; COc1cccc(CC(=O)Nc2ccc(-c3ccncc3)cc2)c1 ; COc1cc(NC(=O)Cc2ccccc2Cl)ccc1-c1ccncc1 ; COc1cc(NC(=O)Cc2cc(F)ccc2Cl)ccc1-c1ccncc1]

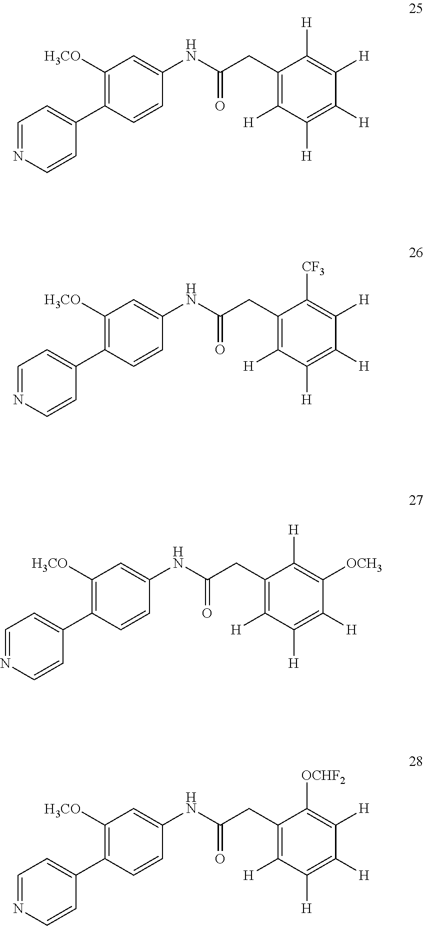 [SMILES: COc1cc(NC(=O)Cc2ccccc2)ccc1-c1ccncc1 ; COc1cc(NC(=O)Cc2ccccc2C(F)(F)F)ccc1-c1ccncc1 ; COc1cccc(CC(=O)Nc2ccc(-c3ccncc3)c(OC)c2)c1 ; COc1cc(NC(=O)Cc2ccccc2OC(F)F)ccc1-c1ccncc1]

16. Composition for use according to claim 10, wherein said composition is suitable for intraocular injection.
